(19) United States
(12) Patent Application Publication
(10) Pub. No.: US 2024/0018176 A1
(43) Pub. Date: Jan. 18, 2024

(54) BIOACTIVE PROCESS FOR BIOGENERATION OF FUNCTIONAL CARBOHYDRATES FROM DAIRY BY-PRODUCTS

(71) Applicant: The Royal Institution For The Advancement Of Learning/McGill University, Montréal (CA)

(72) Inventors: Salwa Karboune, Dollard des Ormeaux (CA); Rami Bahlawan, Verdun (CA); Muriel Yok Kam Wong Min, Port-Louis (MU)

(21) Appl. No.: 18/319,879

(22) Filed: May 18, 2023

Related U.S. Application Data
(60) Provisional application No. 63/364,975, filed on May 19, 2022.

(51) Int. Cl. C07H 3/06; A23C 23/00

(52) U.S. Cl. CPC C07H 3/06; A23C 2220/204; A23C 23/00

(57) ABSTRACT

It is provided a process for producing lactosucrose from a source of lactose such as whey permeate and a source of sucrose as well as a source of both lactose/sucrose such as chocolate milk to enzymatically generating in situ lactosucrose, and fructosylate phenolic compounds contained therein, the process comprising contacting the dairy starting material (containing sucrose) with a levansucrase (LS), which can be immobilized on a solid support, selected from Bacillus amyloliquefaciens (ATCC 23350), Gluconobacter oxydans (strain 621H) (LS1), Vibrio natriegens NBRC 15636 (LS2), Novosphingobium aromaticivorans (LS3), and Burkholderia graminis C4D1M (LS4), or a combination thereof.

### CROSS-REFERENCE TO RELATED APPLICATION

The present application is claiming priority from U.S. Provisional Application No. 63/364,975 filed May 19, 2022, the content of which is hereby incorporated by reference in its entirety.

### TECHNICAL FIELD

It is provided a process to biotransform dairy by-products into functional sweetener such as lactosucrose or to biogenerate in situ functional sweetener in dairy products using levansucrase.

### BACKGROUND

In 2016, it was estimated that whey has a worldwide production of 200 million tons per year and increasing. The volume of whey produced is almost equal to that of the processed milk used in cheese production and therefore it is increasing at a rate equal to that of milk volumes which is >2% per year. Furthermore, cheese production has in fact being increasing by 3% annually and every kg of cheese requires about 10 kg of milk and gives rise to around 9 kg of whey. Unfortunately, treating the whey can be expensive and even difficult due to its constantly changing composition and strict legal requirements but without treating it, it poses a serious environmental hazard particularly on aquatic life. In 2008, a spill of acid whey in a body of water in the state of Ohio led to eutrophication due to the nutrient-dense nature of whey, especially its nitrogen and phosphorous content. That in turn caused a depletion of dissolved oxygen and thus the death of more than 5,400 wild animals, mostly fish, and the development of bad odors. Consequently, the treatment and/or valorization of whey is necessary and since it is a source of functional proteins and peptides, lipids, vitamins, minerals, and lactose, it can be transformed into valuable commodities to be utilized in the agri-food, biotechnological, medical, and many other industries.

One traditional use of whey is the usual dilution of its unmodified form with potable water for use as an animal feed for pigs, sheep, and cattle. It provides them with high quality proteins, lactose as energy source, and micronutrients like calcium, phosphorous, sulphur, and water-soluble vitamins. However, excess lactose and minerals can cause harm to the farm animals and thus it must be limited. Another traditional use of whey is spreading it on the land as a fertilizer but that can lead to a build-up of compounds like salts damaging the soil and impacting plant's growth and lifecycle. Crops have been shown to perish due to excessive whey application which causes rapid oxygen consumption in the soil as a result of breaking down the lactose and proteins in the whey. The whey can also reduce the soil's redox potential and lead to the solubilization of the Fe and Mn present in the soil and thus potentially contaminating ground water supplies. Moreover, a substantial amount of salt is added alongside whey increasing the salinity of the soil and reducing crop yield. If acid whey is added, then it also lowers the pH value damaging the soil. Both the aforementioned uses have drawbacks in terms of the volumes and high transportation costs, so they are impractical considering how much whey is being manufactured nowadays. More modern used of whey as a whole include its use to make beverages with examples including fruit juices and carbonated soft drinks mixed with whey in addition to low alcoholic beverages (<1% alcohol content), whey beer, whey wine, and whey champagne. The alcoholic beverages can be synthesized through the fermentation of whey alongside additives such as sucrose and malt with Kluyveromyces fragilis or Saccharomyces lactis. Other products include whey cheeses, such as Ricotta and Mysost, whey butter, and whey cream. Unfortunately, all the aforementioned products do not have a widespread commercial appeal and thus do not present an effective manner to treat the large quantities of the manufactured whey. Finally, the whey can be spray dried to make whey powder which extends its shelf life for possible transportation and further processing. The whey powder can also be used as an animal feed in the form of dairy nuts when mixed with molasses or soya flour but can also be used in human foods such as ice cream, baked goods, sauces, and so on.

Focusing on the whey components separately, whey proteins tend to come to mind due to their functional properties and thus can be used in the food industry as emulsifiers, gelling agents, foaming agents, and so on. Whey protein is actually used in several products such as soups, salad dressings, processed meats, dairy, and baked goods. Whey proteins are usually separated through various physicochemical processes such as membrane separation (ultrafiltration or diafiltration) and then treated to provide whey protein concentrate (WPC), whey protein isolate (WPI) that has a higher content of protein and almost no lactose and fat, and lastly whey hydrolysate through the enzymatic hydrolysis of WPC and/or WPI. This entire filtration process leaves behind another liquid known as whey permeate which is primarily made of lactose (75-85% w/w) and thus also possess a high BOD making its disposal problematic. Fortunately, it can be crystalized and purified to provide lactose or can be fermented to form ethanol, single cell protein, yeast extract, bioplastics, glycerol, and other organic compounds. Those physicochemical processes can also be applied to milk, such as skimmed milk, to again produce a protein portion and another portion known as milk permeate, which contains a much lower lactose content (4.7% w/w) in comparison to the whey permeate (Boer, 2014).

Levansucrase (LS, EC 2.4.1.10) is a fructosyltransferase that is garnering higher interest due its ability to synthesize novel prebiotics which promote intestinal health. LS is capable of transferring a fructose unit from a fructosyl donor molecule to a fructosyl acceptor molecule resulting in the production of various products. One notable compound where LS offers a potential attractive catalytic activity for its production is lactosucrose, which requires the use of sucrose as a fructosyl donor and lactose as a fructosyl acceptor. Lactosucrose has been increasing in popularity due to its prebiotic and technofunctional properties. Unfortunately, low quantities of lactosucrose exist naturally in foods and thus an efficient biocatalytic system is needed for its synthesis. Sucrose is the most used sweetener in the food industry, and lactose is abundant due to several million tons of it being generated in the dairy by-products known as whey and milk permeate.

It is thus highly desired to be provided with means to biotransform whey and produce functional products such as lactosucrose.

### SUMMARY

It is provided a process for producing lactosucrose from a dairy starting material, the process comprising contacting a source of lactose and a source of sucrose with a levansucrase (LS) selected from Bacillus amyloliquefaciens (ATCC 23350), Gluconobacter oxydans (strain 621H) (LS1), Vibrio natriegens NBRC 15636 (LS2), Novosphingobium aromaticivorans (LS3), and Burkholderia graminis C4D1M (LS4), or a combination thereof, producing lactosucrose.

In an embodiment, the LS is Bacillus amyloliquefaciens (ATCC 23350), Gluconobacter oxydans (strain 621H) (LS1), Vibrio natriegens NBRC 15636 (LS2), or a combination thereof.

In a further embodiment, the LS is Vibrio natriegens NBRC 15636 (LS2).

In another embodiment, the process described herein further comprises producing fructooligosaccharides (FOSs).

In an embodiment, the FOSs are kestose, nystose, fructosyl nystose, or a combination thereof.

In another embodiment, the levansucrase further transfructosylate phenolic compounds from the dairy starting material.

In an embodiment, the phenolic compounds are catechin, gallic acid, caffeic acid, and/or epicatechin.

In another embodiment, the process described herein further comprises producing oligomers and polysaccharides.

In an embodiment, the oligomers have a molecular mas between 5-20 kDa.

In a further embodiment, the polysaccharides have a molecular weight of up to 5000 kDa.

In an embodiment, the polysaccharides are levans.

In a supplemental embodiment, the source of lactose is a dairy product.

In an embodiment, the dairy product is a lactose enriched mixture, a whey permeate (WP), or a milk permeate (MP).

In another embodiment, the dairy starting material is milk, smoothie, yogurt, high protein dairy mixture, or ice cream.

In an embodiment, the milk is a flavored milk or chocolate milk.

In another embodiment, the source of sucrose is white sugar, maple syrup, or a combination thereof.

In a further embodiment, the LS has a higher transfructosylation activity then hydrolysis activity.

In another embodiment, the LS is immobilized on a solid support.

In a particular embodiment, the solid support is a glyoxyl agarose support.

In an embodiment, the glyoxyl agarose support is further modified with iminodiacetic acid (IDA), IDA-Cu, and triethylamine (TEA).

In another embodiment, the solid support is Relizyme™ EP403 or Sepabeads® EC-EP/S.

In an embodiment, the LS is stabilized post immobilization on the solid support by exposing the LS immobilized on the support to high the pH conditions.

In another embodiment, the LS immobilized on the support further comprises a polyaminated crosslinker (PEI).

It is also provided a kit comprising a solid support and a levansucrase (LS) selected from Gluconobacter oxydans (strain 621H) (LS1), Vibrio natriegens NBRC 15636 (LS2), Novosphingobium aromaticivorans (LS3), and Burkholderia graminis C4D1M (LS4), or a combination thereof.

In an embodiment, the solid support is Relizyme™ EP403/IDA-Cu and the LS is V. natriegens LS2.

In a further embodiment, the solid support Relizyme™ EP403/IDA-Cu and the LS is G. oxydans LS1.

It is additionally provided an enriched dairy product comprising a levansucrase (LS) selected from Gluconobacter oxydans (strain 621H) (LS1), Vibrio natriegens NBRC 15636 (LS2), Novosphingobium aromaticivorans (LS3), and Burkholderia graminis C4D1M (LS4), or a combination thereof and a source of sucrose.

In an embodiment, the product is enriched in lactosucrose.

In a particular embodiment, the product is enriched in fructooligosaccharides (FOSs).

### BRIEF DESCRIPTION OF THE DRAWINGS

Reference will now be made to the accompanying drawings.

FIG. 1 illustrates the chemical structure of lactosucrose.

FIG. 2 illustrates time course for hydrolysis vs transfructosylation extent in the presence of lactose (L)/sucrose, in whey permeate (WP)/sucrose, and milk permeate (MP)/sucrose for (a) G. oxydans LS1, (b) V. natriegens LS2, (c) N. aromaticivorans LS3, and (d) B. graminis LS4 levansucrase (LS): extent of sucrose hydrolysis (▪); extent of sucrose transfructosylation (▪); extent of lactose hydrolysis (▪); extent of lactose transfructosylation (▪).

FIG. 3 illustrates MS-MS fragmentation spectra of biotransformation end-products for (a) kestose, (b) lactosucrose, (c) nystose, and (d) fructosyl nystose.

FIG. 4 illustrates biotransformation end-products in the presence of lactose (L)/sucrose, whey permeate (WP)/sucrose, and milk permeate (MP)/sucrose for (a) G. oxydans LS1, (b) V. natriegens LS2, (c) N. aromaticivorans LS3, and (d) B. graminis LS4 levansucrase (LS).

FIG. 5 illustrates the maximum bioconversion yields of lactosucrose and kestose in the reaction system catalyzed in the presence of lactose (L)/sucrose, whey permeate (WP)/sucrose, and milk permeate (MP)/sucrose for each levansucrase (LS).

FIG. 6 illustrates oligomer (5-20 kDa) and levan (up to 5000 kDa) production in the presence of lactose (L)/sucrose, whey permeate (WP)/sucrose, and milk permeate (MP)/sucrose for each levansucrase (LS).

FIG. 7 illustrates G. oxydans LS1 immobilization on iminodiacetic acid (IDA), IDA-Cu, and triethylamine (TEA) glyoxyl agarose supports.

FIG. 8 illustrates post-immobilization treatments (high pH and polyethylenimine (PEI)) for G. oxydans LS1 and V. natriegens LS2 immobilized on Relizyme™ EP403 functionalized with iminodiacetic acid (IDA)-Cu.

FIG. 9 illustrates the effect of post-immobilization treatments (high pH and polyethylenimine (PEI)) on the thermal stability of G. oxydans LS1 and V. natriegens LS2 immobilized on Relizyme™ EP403 functionalized with iminodiacetic acid (IDA)-Cu.

FIG. 10 illustrates quantified biotransformation end-products for (a) G. oxydans LS1 and (b) V. natriegens LS2 immobilized on Relizyme™ EP403 functionalized with iminodiacetic acid (IDA)-Cu in the presence of lactose (L)/sucrose, whey permeate (WP)/sucrose, and milk permeate (MP)/sucrose.

FIG. 11 illustrates maximum bioconversion yields of lactosucrose and kestose in the reaction systems catalyzed by G. oxydans LS1 and V. natriegens LS2 immobilized on Relizyme™ EP403 functionalized with iminodiacetic acid (IDA)-Cu in the presence of lactose (L)/sucrose, whey permeate (WP)/sucrose, and milk permeate (MP)/sucrose.

FIG. 12 illustrates contour plots of V. natriegens LS2 in its (a₁₋₄) free form and (b₁₋₆) immobilized form on Relizyme™ EP403 functionalized with iminodiacetic acid (IDA)-Cu. All reactions maintained the same enzyme units (5 U/ml), temperature (45° C.), and buffer (50 mM ammonium bicarbonate; pH of 8). The two variables were the reactions time (3-15 h) and ratio of lactose to sucrose (0.5-2.0).

FIG. 13 illustrates responses of the optimal conditions for lactosucrose production with V. natriegens LS2 in its free form and immobilized form on Relizyme™ EP403 functionalized with iminodiacetic acid (IDA)-Cu with 5-time reusability.

FIG. 14 illustrates the time course of the bioconversion of catechin, gallic acid and caffeic acid with LS1 from G. oxydans (strain 621H), LS2 from V. natriegens NBRC 15636, LS3 from N. aromaticivorans, and LS4 from B. graminis C4D1.

FIG. 15 illustrates the number of fructosyl groups acquired by phenolic compounds (F: Fructosyl groups acquired).

FIG. 16 illustrates MS-MS spectra of fructosylated catechin products and their corresponding possible structures obtained from reaction with LS3 from N. aromaticivoran.

FIG. 17 illustrates that sucrose conversion of reactions with LS1 from G. oxydans (strain 621H), LS2 from V. natriegens NBRC 15636, LS3 from N. aromaticivorans, and LS4 from B. graminis C4D1 at pH 6.6 and different temperatures.

FIG. 18 illustrates the effect of milk and cocoa powder on LS1-catalyzed reactions (S: Sucrose concentration in Molar, M: Milk powder concentration in % w/v; C: Cocoa powder concentration in % w/v).

FIG. 19 illustrates the effect of milk and cocoa powder on LS2-catalyzed reactions (S: Sucrose concentration in Molar, M: Milk powder concentration in % w/v; C: Cocoa powder concentration in % w/v).

FIG. 20 illustrates the effect of milk and cocoa powder on LS3-catalyzed reactions (S: Sucrose concentration in Molar, M: Milk powder concentration in % w/v; C: Cocoa powder concentration in % w/v).

FIG. 21 illustrates the effect of milk and cocoa powder on LS4-catalyzed reactions (S: Sucrose concentration in Molar, M: Milk powder concentration in % w/v; C: Cocoa powder concentration in % w/v).

### DETAILED DESCRIPTION

It is provided a process for producing lactosucrose from a dairy starting material, the process comprising contacting the dairy starting material comprising lactose and sucrose with a levansucrase (LS) selected from Gluconobacter oxydans (strain 621H) (LS1), Vibrio natriegens NBRC 15636 (LS2), Novosphingobium aromaticivorans (LS3), Bacillus amyloliquefaciens (ATCC 23350) and Burkholderia graminis C4D1M (LS4), or a combination thereof, producing lactosucrose.

The enzymatic activity of LS varies depending on its microbial source. Four LS strains from Gluconobacter oxydans (strain 621H) (LS1), Vibrio natriegens NBRC 15636 (LS2), Novosphingobium aromaticivorans (LS3), and Burkholderia graminis C4D1M (LS4) were selected and examined in three different reaction systems lactose/sucrose, whey permeate (WP)/sucrose, and milk permeate (MP)/sucrose. The catalytic efficiency of all 4 LSs demonstrated a higher transfructosylation activity as opposed to the hydrolytic one (1.29-7.28), with the sole exception of V. natriegens LS2 in the presence of MP/sucrose (0.16). Moreover, the bioconversion end-products, such as lactosucrose and fructooligosaccharides (FOSs), exhibited varying production time courses and profiles depending on the type of LS and starting material used. The V. natriegens LS2 resulted in the highest bioconversion giving rise to 328 g/L and 251 g/L of lactosucrose with lactose/sucrose and WP/sucrose, respectively. Contrary to other LSs, N. aromaticivorans LS3 synthesized the lowest amounts of lactosucrose of 39.7 g/L with lactose/sucrose, 30.6 g/L with WP/sucrose, and 2.0 g/L with MP/sucrose. N. aromaticivorans LS3 showed a higher product specificity toward the synthesis of FOSs, in particular kestose, nystose, and fructosyl nystose, in all investigated reaction systems. Additionally, G. oxydans LS1 was the sole LS that generated levan polymers when using lactose/sucrose and MP/sucrose (0.71 g/L).

Consequently, the immobilization of G. oxydans LS1 and V. natriegens LS2 by multipoint covalent attachment was studied with the main focus of augmenting the enzyme's preference for transfructosylation over hydrolysis through a modification of its microenvironment in addition to improving the thermal stability. Modified glyoxyl-agarose, Sepabeads® EC-EP/S, and Relizyme™ EP403/S were chosen as solid supports. It was found that the iminodiacetic acid/Cu (IDA/Cu) Relizyme™ EP403/S achieved the highest retained activity with 55% for G. oxydans LS1 and 98% for V. natriegens LS2. The greatest thermal stability (a stabilization factor of 53 at 50° C.) was experienced by V. natriegens LS2 immobilized onto Relizyme™ EP403/S-IDA/Cu that had been treated with a post-immobilization step involving a high pH incubation. Additionally, the process of immobilizing both LSs was found to provide a great modulation of the enzyme's microenvironment with transfructosylation over hydrolysis ratios being higher than in their free forms, particularly for V. natriegens LS2 with ratios for the sucrose/lactose reaction of 3.51 in its free form in comparison to 1637, 5, and 4, respectively, for glycoxyl agarose-IDA/Cu, Sepabeads® EC-EP/S-IDA/Cu, and Relizyme™ EP403/S-IDA/Cu. The V. natriegens LS2 immobilized on Relizyme™ EP403/S-IDA/Cu was found to provide the highest produced lactosucrose amounts with 96, 86, and 35 g/L respectively for lactose/sucrose, WP/sucrose, and MP/sucrose.

Ultimately, the V. natriegens LS2 was chosen for the optimization of the lactosucrose synthesis using the WP/sucrose substrate combination. The lactose (WP)/sucrose ratio and incubation time were the factors selected for examination and optimization using response surface methodology with both free and Relizyme™ EP403/S-IDA/Cu immobilized forms of V. natriegens LS2. The optimal lactose/sucrose ratio and incubation time were determined to be, respectively, 0.59 and 3.12 h for the free V. natriegens LS2 and 0.50 and 3.08 h for the Relizyme™ EP403/S-IDA/Cu immobilized V. natriegens LS2. Furthermore, the Relizyme™ EP403/S-IDA/Cu immobilized V. natriegens LS2 was efficiently reused in 3 consecutive reactions, doubling the amount of produced lactosucrose compared to that obtained with the free V. natriegens LS2.

The food industry generates a significant quantity of by-products and waste; therefore, the prevention of waste generation or its exploitation is one of the main objectives of environmental management and industries. Yogurt, milk, ice cream, and so on are just some of the few everyday consumed products from the dairy industry. However, the dairy industry also generates a green-yellow liquid by-product, known as whey, as part of the production of cheese or casein from milk. The composition and physicochemical characteristics of whey are primarily dependent on the milk used and its animal source, diet, health, and lactation stage. However, whey always has a relatively high organic load dictated by the high biochemical oxygen demand (BOD) and chemical oxygen demand (COD). Its high organic load is primarily due to the presence of milk carbohydrates followed by proteins in addition to fats, minerals, and other suspended solids. Whey makes up 85-95% of the milk volume and keeps around 55% of the nutrients from the milk. 93% of the total whey volume is water followed by total solids that are divided into 66-77% (w/w) lactose, 8-15% (w/w) proteins, and 7-15% (w/w) minerals (mainly sodium, potassium, calcium, and magnesium salts) with trace amounts of fats, metals, such as zinc and copper, lactic acid, citric acid, non-protein nitrogen compounds, such as urea and uric acid, and B vitamins, such as riboflavin (vitamin B2) that provides whey with its yellow color.

Both whey and milk permeate are recovered upon filtration steps, where the protein portion is isolated. The generation of dairy by-products is increasing in volume where in 2016, it was estimated that whey has a worldwide production of 200 million tons/year and its disposal represents an environmental hazard. Lactose constitutes up to 75-85% (w/w) of whey permeate.

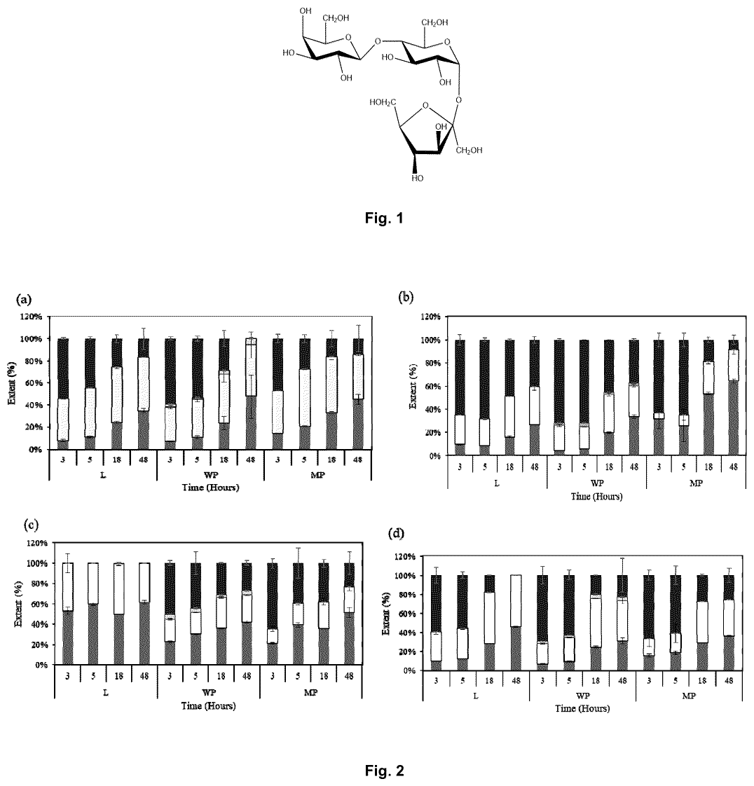

Lactosucrose is composed of a D-galactopyranosyl (Galp) unit linked via a β-1, α-4 glycosidic bond to a D-glucopyranosyl (Glcp) unit that in turn is connected to a D-fructofuranosyl (Fruf) unit via an α-1, β-2 glycosidic bond), making it an isomer to the commonly known sugar raffinose (see structure depicted in FIG. 1). The major difference between the two isomers is that the non-reducing end of raffinose with the galactosyl unit is in the α type configuration while that of lactosucrose is in the β type configuration. Comparing it to sucrose (2000 g/L at 25° C.), lactosucrose (3670 g/L at 25° C.) has a much higher solubility in water and ˜30% of the relative sweetness thus providing a high-quality taste similar to sucrose when compared to other oligosaccharide sweeteners.

The catalytic efficiency of selected LSs was investigated in the presence of sucrose, lactose/sucrose, WP/sucrose, and MP/sucrose substrates. Sucrose can act as a fructosyl donor and acceptor, while lactose behaves mainly as a fructosyl acceptor. Table 1 shows the total, hydrolytic, and transfructosylation activities of selected LSs. The hydrolytic activity of LS resulted in the release of glucose and fructose, whereas the transfructosylation activity of LS led to the transfer of fructose to an acceptor or to a fructose growing chain, resulting in the formation of lactosucrose, FOSs, and/or levan. The hydrolytic activity was estimated from the free fructose concentration, while the transfructosylation activity was determined by subtracting the concentration of free fructose from that of glucose.

Table 1 shows that B. graminis LS4 exhibited the highest total activity (543.60-805.80 μmol/mg protein·min) when lactose, WP, or MP were used as acceptor substrates; it was also the only LS to provide a higher total activity with lactose source substrates than when sucrose was used by itself. Contrary to B. graminis LS4, a higher total activity was exhibited by G. oxydans LS1 (3.60 μmol/mg protein. min), V. natriegens LS2 (78.02 μmol/mg protein·min), and N. aromaticivorans LS3 (142.31 μmol/mg protein·min) when sucrose was the sole substrate than when it was added alongside lactose, WP, or MP (0.31-0.46, 41.25-68.16, and 46.08-63.29 μmol/mg protein·min, respectively).

TABLE 1
Catalytic efficiency of each levansucrase (LS) using sucrose, lactose/sucrose,
whey permeate (WP)/sucrose, and milk permeate (MP)/sucrose

| | | Total | Hydrolytic | Transfructosylation | |
|---|---|---|---|---|---|
| | | Activity | Activity | Activity | Transfructosylation/ |
| | | (μmol/mg | (μmol/mg | (μmol/mg | Hydrolysis |
| Enzyme | Substrate | protein · min)^{a} | protein · min)^{b} | protein · min)^{c} | Ratio^{d} |
| G. oxydans | Sucrose | 3.60 (±0.01) | 1.04 (±0.01) | 2.57 (±0.01) | 2.46 |
| LS1 | | | | | |
| | Lactose/Sucrose | 0.31 (±0.01) | 0.05 (±0.003) | 0.27 (±0.004) | 5.87 |
| | WP/Sucrose | 0.32 (±0.01) | 0.05 (±0.001) | 0.28 (±0.01) | 5.77 |
| | MP/Sucrose | 0.46 (±0.02) | 0.12 (±0.01) | 0.35 (±0.01) | 6.74 |
| V. natriegens | Sucrose | 78.02 (±4.24) | 53.25 (±3.10) | 24.77 (±0.11) | 0.47 |
| LS2 | | | | | |
| | Lactose/Sucrose | 68.16 (±0.01) | 21.79 (±0.01) | 46.38 (±0.004) | 2.13 |
| | WP/Sucrose | 41.25 (±0.52) | 4.98 (±0.23) | 36.27 (±0.29) | 7.28 |
| | MP/Sucrose | 68.05 (±0.04) | 58.66 (±0.04) | 9.39 (±0.001) | 0.16 |
| N. aromaticivorans | Sucrose | 142.31 (±10.30) | 68.80 (±5.40) | 73.51 (±5.30) | 1.07 |
| LS3 | | | | | |
| | Lactose/Sucrose | 46.08 (±0.73) | 16.53 (±2.50) | 29.55 (±1.77) | 1.79 |
| | WP/Sucrose | 63.29 (±0.87) | 17.40 (±1.31) | 45.89 (±0.41) | 2.64 |
| | MP/Sucrose | 59.56 (±0.08) | 22.20 (±2.81) | 37.36 (±2.73) | 1.68 |
| B. graminis | Sucrose | 101.19 (±2.30) | 46.60 (±1.21) | 54.60 (±3.21) | 1.17 |
| LS4 | | | | | |
| | Lactose/Sucrose | 543.60 (±17.29) | 98.16 (±6.93) | 445.44 (±10.36) | 4.54 |
| | WP/Sucrose | 623.44 (±19.12) | 113.87 (±2.06) | 509.57 (±17.06) | 4.48 |
| | MP/Sucrose | 805.80 (±32.85) | 352.07 (±15.58) | 453.73 (±17.27) | 1.29 |

^{a}Total activity was calculated by taking the slope of glucose in mmol/ml · min and multiplying it by the total reaction volume and dividing that by the enzyme content.
^{b}Hydrolytic activity was calculated by taking the slope of fructose in mmol/ml · min and multiplying it by the total reaction volume and dividing that by the enzyme content.
^{c}Transfructosylation activity was calculated by taking the difference between the total activity and hydrolytic activity.
^{d}The ratio of transfructosylation to hydrolysis of each LS

When comparing the lactose sources, the use of MP resulted in the highest total activity for G. oxydans LS1 and B. graminis LS4 with V. natriegens LS2 also falling under the same trend as it had very close total activities in the presence of lactose or MP. However, N. aromaticivorans LS3 had the highest total activity when WP was present. Substrate activation was observed with B. graminis LS4, particularly with MP (805.80 μmol/mg protein·min) followed by WP (623.44 μmol/mg protein·min) instead of lactose. Such results can be attributed to the protective effect of dairy by-product components on the enzyme activity and stability. Looking through the literature, there was a lack of reported LS total activity values in the presence of a lactose source. As provided herein, the reaction conditions were modulated in order to favor the transfructosylation reaction over the hydrolytic one. Looking at other LSs from different microbial sources, it was found that the total activity expressed by Paraburkholderia graminis LS (7.7 μmol/mg protein·min), Streptococcus salivarius LS (31.9 μmol/mg protein·min), and Beijernickis indica LS (12.5 μmol/mg protein·min) were lower than the total activity values exhibited by V. natriegens LS2, N. aromaticivorans LS3, and B. graminis LS4 in the presence of sucrose, lactose/sucrose, WP/sucrose, and MP/sucrose. However, they were higher than the total activity values exhibited by G. oxydans LS1 with sucrose, lactose/sucrose, WP/sucrose, and MP/sucrose.

It can also be seen from the results that most of LSs exhibited higher transfructosylation activity than hydrolytic one. Despite G. oxydans LS1 having the lowest total activity among the enzymes with all substrate combinations, its transfructosylation to hydrolysis ratios were some of the highest. Indeed, the transfructosylation to hydrolysis ratio of G. oxydans LS1 was estimated at 2.46, 5.87, 5.77, and 6.74 when sucrose, lactose/sucrose, WP/sucrose, and MP/sucrose were used as substrates, respectively. These results reveal the high catalytic efficiency of G. oxydans LS1 towards the transfructosylation reaction. Contrary to other selected LSs, V. natriegens LS2 exhibited the lowest transfructosylation to hydrolysis ratios (0.16-2.13) in the presence of sucrose, lactose/sucrose, and MP/sucrose substrates, but achieved the highest observed ratio of 7.28 when WP/sucrose substrate was used. Additionally, the ratio of transfructosylation to hydrolysis appeared to have increased when sucrose was used alongside a lactose source in all cases except for V. natriegens LS2 in the presence of MP/sucrose substrate (0.16). The substrate combination of WP/sucrose provided the highest transfructosylation to hydrolysis ratio for V. natriegens LS2 (7.28) and N. aromaticivorans LS3 (2.64). Furthermore, the highest ratio for B. graminis LS4 (4.48-4.54) was achieved in the presence of lactose/sucrose and WP/sucrose substrates. However, G. oxydans LS1 exhibited the highest ratio of transfructosylation to hydrolysis in the presence of MP/sucrose (6.74).

As provided herewith, the transfructosylation to hydrolysis ratios of N. aromaticivorans LS3 (1.07), B. graminis LS4 (1.17) and G. oxydans LS1 (2.46) in the presence of only sucrose were within the reported range in the literature. On the other hand, the obtained ratio for V. natriegens LS2 ratio (0.47) was lower. However, the addition of lactose substrate source enhanced the transfructosylation to hydrolysis ratio for most of studied LSs.

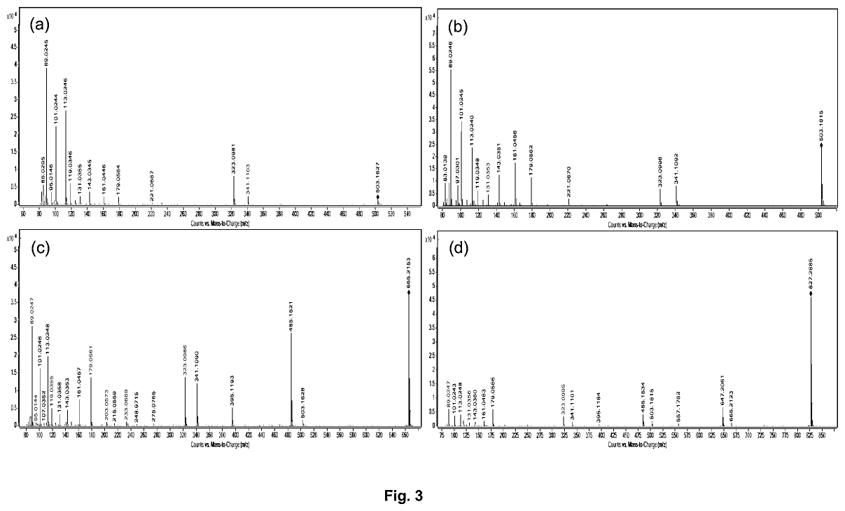

Over the time course of the 48 h biotransformation reactions of lactose/sucrose, sucrose/WP, and sucrose/MP, a shift in the thermodynamics of the reactions may occur, affecting the extents of transfructosylation and hydrolysis (FIG. 2). Focusing first on sucrose biotransformation, the lactose/sucrose reaction catalyzed by G. oxydans LS1 exhibited a higher transfructosylation extent than hydrolysis one throughout the entire reaction time course. However, the G. oxydans LS1-catalyzed WP/sucrose and MP/sucrose biotransformation reactions showed the dominance of the sucrose transfructosylation extent at the early stage; as the reaction progressed, the hydrolysis extent became greater, reaching maximum values at 48 h.

The V. natriegens LS2-catalyzed lactose/sucrose reaction displayed a pattern where the transfructosylation extent was higher than that of hydrolysis over the entire time course of the reaction. However, the transfructosylation extent of the WP/sucrose reaction catalyzed by V. natriegens LS2 was initially greater than that of hydrolysis, but then at the end of the 48 h reaction time, the extent of hydrolysis became greater. Contrary to V. natriegens LS2-catalyzed lactose/sucrose and WP/sucrose reactions, the hydrolysis extent of sucrose was predominant in the MP/sucrose reaction throughout the entire reaction time course. This is in agreement with the catalytic efficiency results shown in Table 1 where the hydrolytic activity of V. natriegens LS2 was greater than its transfructosylation when MP/sucrose was used. N. aromaticivorans LS3-catalyzed lactose/sucrose, WP/sucrose, and MP/sucrose reactions appeared to display more or less similar hydrolysis and transfructosylation extents of sucrose at the early stage, but then the hydrolysis extent took over for the remainder of the reaction time courses. B. graminis LS4-catalyzed lactose/sucrose, WP/sucrose, and MP/sucrose reactions all exhibited a higher transfructosylation extent than hydrolysis one throughout the entire reaction time course. The results (FIG. 2) also show that the highest extent of transfructosylation of sucrose was achieved at around 18 h with all 4 LSs with the sole exception being at 5 h with G. oxydans LS1 for the MP/sucrose reaction. G. oxydans LS1 in fact delivered the highest sucrose transfructosylation extents for lactose/sucrose, WP/sucrose, and MP/sucrose reactions, estimated at 54.66%, 45.73%, and 47.08%, respectively.

Examining lactose biotransformation, no hydrolysis took place with all 4 LSs when lactose/sucrose and MP/sucrose substrates were used. Only a limited hydrolysis extent of lactose (<5%) was observed at time 0 h in the presence of WP/lactose substrate (FIG. 2). No significant change in the hydrolysis extent of lactose was obtained throughout the reactions, revealing that LSs did not contribute to the initial observed hydrolysis of lactose in the WP/lactose reaction. For the extent of lactose transfructosylation, a similar pattern was observed for G. oxydans LS1 and B. graminis LS4, where the transfructosylation extent started off high to reach maximum values at 3-5 h but decreased steadily thereafter over the reaction time course. The lactose/sucrose reaction-catalyzed by N. aromaticivorans LS3 showed no significant transfructosylation of lactose. However, the extent of lactose transfructosylation in the N. aromaticivorans LS3-catalyzed WP/sucrose and MP/sucrose reactions varied between 23.72-29.81% and 18.53-34.79%, respectively, with a peak at 18 h. The V. natriegens LS2 showed a high extent of lactose transfructosylation (56.82-72.32%) at the start (3 h) of the lactose/sucrose, WP/sucrose, and MP/sucrose reactions that decreased over the time course of the reactions.

The results (FIG. 2) also indicate that the use of G. oxydans LS1 and V. natriegens LS2 resulted in the highest extents of lactose transfructosylation at 3 h and 5 h in all their investigated reactions. Furthermore, B. graminis LS4 had its highest lactose transfructosylation extent at 5 h in lactose/sucrose reaction and 3 h in WP/sucrose and MP/sucrose reactions. The highest extents of lactose transfructosylation were obtained with V. natriegens LS2 in lactose/sucrose (61.22%, 5 h) and WP/sucrose (72.32%, 3 h) reactions. The highest lactose transfructosylation extent of 60.93% for the MP/sucrose reaction was achieved with B. graminis LS4 at 3 h.

The end-product profiles of the biotransformation reactions were characterized by Q-TOF-MS. FIG. 3 shows the MS-MS spectra conducted in order to confirm the identity of the selected end-products where the fragmented compounds in the samples were compared to the fragmented compounds in the standards. The precursor ion for kestose and lactosucrose was m/z 503 and they shared a good portion of the resulting fragments with the major ones being m/z 341, 323, 179, 161, 119, and 101. The most easily identifiable fragments were m/z 341 for the sucrose unit formed by losing a fructose or galactose unit, m/z 323 for a hexose derivate, m/z 179 for possibly fructose, m/z 161 for a hexose structure of the monosaccharides, m/z 119 and 101 as fragments of glucose. The main differences were seen at the lower end with m/z 85 and 95 for kestose but m/z 83 and 97 for lactosucrose. The nystose had a precursor ion of m/z 665 with major fragments at m/z 503 and 485, while fructosyl nystose had a precursor ion at m/z 827 that fragments into m/z 665 and 647 with the remaining fragments matching those that can be seen in kestose.

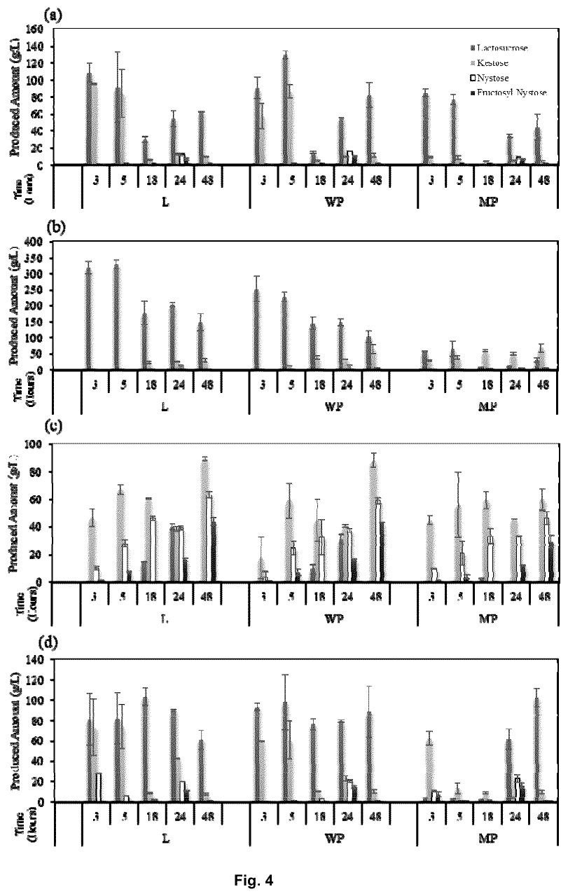

FIG. 4 demonstrates the end-product profiles of lactose/sucrose, WP/sucrose, and MP/sucrose reactions for each LS. The G. oxydans LS1 was able to catalyze the synthesis of both lactosucrose and kestose, achieving the highest contents within the first 5 h of lactose/sucrose, WP/sucrose, and MP/sucrose reactions. In the lactose/sucrose and WP/sucrose reactions, G. oxydans LS1 synthesized high amounts of both of these compounds with a predominant product specificity towards lactosucrose (108.4-129.5 g/L) over kestose (86.0-94.6 g/L), while the MP/sucrose reaction resulted in the synthesis of higher amount of lactosucrose (84.3 g/L) than kestose (9.2 g/L).

B. graminis LS4 was able to produce a constant amount of lactosucrose throughout the time course of WP/sucrose (97.8 g/L) and lactose/sucrose (103.1 g/L) reactions up to 5 h and 18 h, respectively. The production of kestose by B. graminis LS4 in lactose/sucrose and WP/sucrose reactions took primarily place within the first 5 h of reaction, leading to a maximum amount of 73.5 and 60.0 g/L, respectively. The B. graminis LS4-catalyzed MP/sucrose biotransformation reaction favored the kestose synthesis at the start, with 61.9 g/L produced at 3 h, which decreased over the time course of the reaction. A quantity of lactosucrose of 61.3 g/L eventually appeared at 24 h with the B. graminis LS4 in the MP/sucrose reaction and continued to increase to reach 102.0 g/L at 48 h. Nonetheless, an overall decrease in both lactosucrose and kestose quantities was displayed in the reactions catalyzed by G. oxydans LS1 and B. graminis LS4, which was attributed to the potential hydrolysis, possibly with lactosucrose, and/or to the elongation of end-products into oligomers and polymers, which was suggested with the emergence of nystose and fructosyl nystose.

The V. natriegens LS2-catalyzed lactose/sucrose and WP/sucrose reactions have primarily produced lactosucrose and very little kestose showing a high affinity of this LS towards lactose as an acceptor substrate (FIG. 4). The highest amount of lactosucrose was estimated to be 327.6 g/L at 5 h with lactose/sucrose reaction and 250.5 g/L at 3 h with WP/sucrose reaction. Also, focusing on V. natriegens LS2, lactosucrose (63.7 g/L) over kestose (35.9 g/L) production was favored in the MP/sucrose reaction at the start but then kestose production took over at 18 h till the end of the reaction time (66.3 g/L). These results support the trends observed in FIG. 3 in which a high lactose transfructosylation extent was obtained at the first 5 h and shifted thereafter towards a high sucrose transfructosylation extent. The predominance of the hydrolysis in the V. natriegens LS2-catalyzed MP/sucrose reaction as previously shown in Table 1 can explain the overall small amount of end-products observed in this reaction.

The results (FIG. 4) also show that N. aromaticivorans LS3 displayed an opposite pattern compared to other LSs as there was little lactosucrose and primarily kestose and other FOSs being formed. Despite Table 1 showing a relatively high total activity of N. aromaticivorans LS3 in the presence of all lactose sources, N. aromaticivorans LS3 appeared to have a low acceptor specificity towards lactose. This is further supported by the observed low lactose transfructosylation extent (FIG. 2) where it had the overall lowest lactose transfructosylation values among the LSs, especially in the lactose/sucrose reaction. Consequently, N. aromaticivorans LS3 was able to produce the highest amount of kestose, nystose, and fructosyl nystose, corresponding respectively to 89.1, 62.9, and 43.4 g/L with lactose/sucrose, 88.1, 58.6, and 42.8 g/L with WP/sucrose, and 59.9, 46.0, and 29.1 g/L with MP/sucrose. However, the highest amount of lactosucrose produced by N. aromaticivorans LS3 was estimated at 39.7 g/L with lactose/sucrose, 30.6 g/L with WP/sucrose, and 2.0 g/L with MP/sucrose.

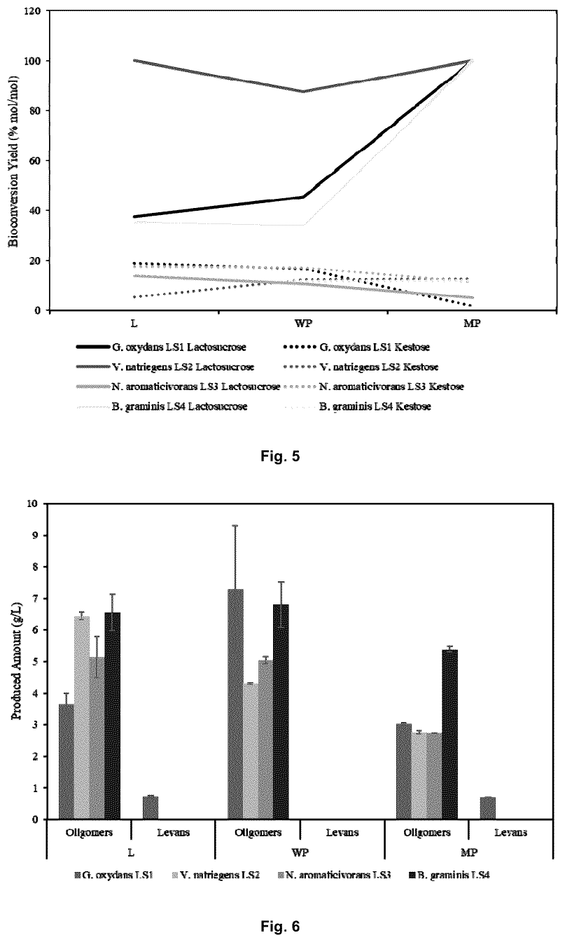

The initial lactose concentrations in lactose/sucrose and WP/sucrose biotransformation systems were comparable. However, as MP was composed of a lower lactose concentration, it was difficult to reach the desired 0.45M lactose concentration without a drying step for removing the water. Therefore, to compare the production efficiency, the bioconversion yields of lactosucrose and kestose were calculated. For each LS and substrate used, the time point where there was a maximum production for each of kestose and lactosucrose was identified (FIG. 5). As a sucrose to lactose ratio of 2:1 was used, the estimated lactosucrose bioconversion yield (calculated based on the initial lactose concentration) was shown to be higher than that of kestose (calculated based on the initial sucrose concentration). FIG. 5 shows that the bioconversion yield of kestose was highest for G. oxydans LS1 with lactose/sucrose (19%). However, G. oxydans LS1 has the lowest kestose bioconversion yield with MP/sucrose (2%), while the other LSs have somewhat similar kestose bioconversion yields with MP/sucrose (11-13%). N. aromaticivorans LS3 has the highest kestose bioconversion yield with WP/sucrose (17%). The highest lactosucrose bioconversion yields with lactose/sucrose and WP/sucrose are provided by V. natriegens LS2 (100% and 88%, respectively). The results also show that, except for N. aromaticivorans LS3, all LSs led to the production of the same maximum lactosucrose bioconversion yield with MP/sucrose.

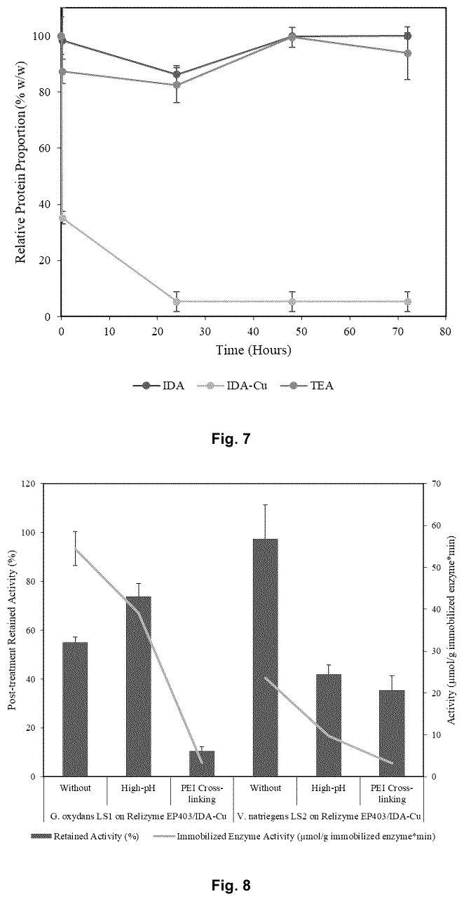

The ability of LSs to produce longer chained oligomers and polysaccharides, particularly levans, was examined using HPSEC. FIG. 6 depicts the amount of oligomers and levans produced by each LS for each substrate combination. All LSs generated oligomers where B. graminis LS4 produced the highest amounts of 6.55 and 5.38 g/L in the presence of lactose/sucrose and MP/sucrose substrates, respectively. G. oxydans LS1 produced the highest amount of oligomers of 7.28 g/L in the WP/sucrose reaction, whereas lower oligomers amounts of 3.02-3.64 g/L were obtained in the lactose/sucrose and MP/sucrose reaction systems. G. oxydans LS1 was the only LS able to produce levans in these lactose/sucrose (0.72 g/L) and MP/sucrose (0.71 g/L) reaction systems, indicating its polymerization ability.

In order to confirm whether lactosucrose has the ability to be a fructosyl acceptor and/or donor, lactosucrose alone or with sucrose was utilized as a substrate in the LS-catalyzed biotransformation reactions. By looking at the percent of converted substrates, it can be seen that both lactosucrose and sucrose/lactosucrose reactions catalyzed by G. oxydans LS1, N. aromaticivorans LS3, and B. graminis LS4 showed increasing bioconversions of substrates up to 85-99%. The percent of converted substrates with V. natriegens LS2 provided a different pattern as it reached its highest value at 3 h but then decreased with reaction time from 46% to 23% with lactosucrose alone and 59% to 51% with the sucrose/lactosucrose.

The aforementioned complete conversion of lactosucrose substrate by G. oxydans LS1, N. aromaticivorans LS3, and B. graminis LS4 is predominantly due to its hydrolysis, particularly with N. aromaticivorans LS3 where it even reached 100% after 48 h. On the other hand, the sucrose/lactosucrose reaction-catalyzed by G. oxydans LS1 showed an extent of transfructosylation (48.7%) that somewhat matched that of hydrolysis (48.9%) at 18 h before the hydrolysis extent took over and dominated at 48 h reaching 63.8%. The higher percent of transfructosylation extent at 18 with G. oxydans LS1 was probably due to the elongation of the growing chain of fructose units as the concentrations of released lactose and fructose were similar in pattern and magnitude in lactosucrose and sucrose/lactosucrose reactions.

The results (Table 2) also show that the transfructosylation extent was higher than that of hydrolysis and was ascending throughout the sucrose/lactosucrose reactions catalyzed by N. aromaticivorans LS3 and B. graminis LS4, achieving 49% and 84%, respectively. The sucrose/lactosucrose reaction catalyzed by B. graminis LS4 exhibited an extent of transfructosylation that was constantly higher than that of hydrolysis, while no increase in either lactosucrose, kestose, nystose or fructosyl nystose was observed. These results suggest that lactosucrose may have acted as a fructosyl acceptor by B. graminis LS4. Lastly, the V. natriegens LS2 reaction with only lactosucrose was able to denote that lactosucrose could be used as a fructosyl donor but the use of lactosucrose as a fructosyl acceptor should be further examined by looking at its possible elongation products. Furthermore, the concentration of the released lactose decreased in both lactosucrose and sucrose/lactosucrose V. natriegens LS2 reactions, while that of lactosucrose increased.

TABLE 2
The catalytic actions of each levansucrase (LS) on lactosucrose and sucrose/lactosucrose substrates.

| | | | Converted | | | | |
|---|---|---|---|---|---|---|---|
| | | Time | Substrate(s) | Transfructosylation | Hydrolysis extent | Released | Release |
| Enzyme | Substrate | (Hours) | (%)^{a, b} | extent (%)^{c, d} | (%)^{e, f} | Lactose (M)^{g} | Fructose (M)^{h} |
| G. oxydans | Lactosucrose | 0 | 0.00 (±0.00) | 0.00 (±0.00) | 0.00 (±0.00) | 0.00 (±0.00) | 0.00 (±0.00) |
| LS1 | | 3 | 67.68 (±3.53) | 43.01 (±2.51) | 24.67 (±1.02) | 0.28 (±0.02) | 0.16 (±0.01) |
| | | 18 | 98.83 (±0.002) | 34.01 (±0.78) | 64.82 (±0.77) | 0.47 (±0.001) | 0.33 (±0.003) |
| | | 48 | 99.53 (±0.01) | 21.00 (±3.51) | 78.53 (±3.49) | 0.45 (±0.01) | 0.36 (±0.00) |
| | Sucrose/ | 0 | 0.00 (±0.00) | 0.00 (±0.00) | 0.00 (±0.00) | 0.00 (±0.00) | 0.00 (±0.00) |
| | Lactosucrose | 3 | 38.25 (±1.38) | 18.46 (±1.12) | 19.79 (±0.26) | 0.21 (±0.001) | 0.15 (±0.001) |
| | | 18 | 97.56 (±0.33) | 48.69 (±0.55) | 48.87 (±0.88) | 0.49 (±0.004) | 0.39 (±0.01) |
| | | 48 | 98.68 (±0.12) | 34.92 (±8.28) | 63.76 (±8.40) | 0.48 (±0.03) | 0.52 (±0.07) |
| V. natriegens | Lactosucrose | 0 | 0.00 (±0.00) | 0.00 (±0.00) | 0.00 (±0.00) | 0.00 (±0.00) | 0.00 (±0.00) |
| LS2 | | 3 | 46.14 (±0.95) | 11.10 (±0.49) | 35.04 (±0.46) | 0.30 (±0.01) | 0.28 (±0.01) |
| | | 18 | 34.95 (±3.67) | 20.30 (±0.81) | 14.66 (±2.86) | 0.15 (±0.01) | 0.11 (±0.01) |
| | | 48 | 23.42 (±2.56) | 30.47 (±0.10) | 0.00 (±2.46) | 0.11 (±0.003) | 0.08 (±0.003) |
| | Sucrose/ | 0 | 0.00 (±0.00) | 0.00 (±0.00) | 0.00 (±0.00) | 0.00 (±0.00) | 0.00 (±0.00) |
| | Lactosucrose | 3 | 58.70 (±1.75) | 12.86 (±0.89) | 45.84 (±0.86) | 0.32 (±0.01) | 0.45 (±0.01) |
| | | 18 | 53.32 (±5.27) | 28.02 (±3.97) | 25.31 (±1.31) | 0.21 (±0.01) | 0.25 (±0.01) |
| | | 48 | 51.31 (±13.35) | 31.20 (±9.22) | 20.11 (±4.13) | 0.18 (±0.03) | 0.20 (±0.03) |
| N. aromaticivorans | Lactosucrose | 0 | 0.00 (±0.00) | 0.00 (±0.00) | 0.00 (±0.00) | 0.00 (±0.00) | 0.00 (±0.00) |
| LS3 | | 3 | 44.85 (±6.65) | 0.19 (±0.70) | 44.66(±5.95) | 0.15 (±0.001) | 0.15 (±0.002) |
| | | 18 | 81.08 (±0.51) | 0.00 (±1.07) | 85.06 (±0.56) | 0.33 (±0.01) | 0.36 (±0.02) |
| | | 48 | 94.66 (±1.40) | 0.00 (±2.79) | 100.00 (±1.39) | 0.38 (±0.07) | 0.43 (±0.08) |
| | Sucrose/ | 0 | 0.00 (±0.00) | 0.00 (±0.00) | 0.00 (±0.00) | 0.00 (±0.00) | 0.00 (±0.00) |
| | Lactosucrose | 3 | 44.87 (±8.72) | 28.34 (±3.04) | 16.54 (±5.68) | 0.01 (±0.002) | 0.21 (±0.05) |
| | | 18 | 63.85 (±3.64) | 33.51 (±0.33) | 30.33 (±3.97) | 0.12 (±0.01) | 0.38 (±0.03) |
| | | 48 | 85.23 (±3.77) | 49.15 (±1.80) | 36.09 (±1.98) | 0.23 (±0.01) | 0.46 (±0.02) |

^{a}Only lactosucrose was a substrate in this reaction and thus the percent of converted substrate was equal to the lactosucrose quantity at a specified time point as a percentage of the initial lactosucrose quantity.
^{b}Both lactosucrose and sucrose were substrates in this reaction and thus the percent of converted substrates was equal to 100 minus the sucrose and lactosucrose quantities at a specified time point as a percentage of the initial sucrose and lactosucrose quantities.
^{c}Only lactosucrose was a substrate in this reaction and the transfructosylation percent was calculated by taking subtracting the fructose quantity from lactose quantity at a specified time point divided by the difference between the lactosucrose quantity at the same specified time point and its initial quantity and multiplying the result by 100.
^{d}Both lactosucrose and sucrose were substrates in this reaction and the transfructosylation percent was calculated by subtracting the percent hydrolysis from the percent of converted substrates at a specified time point.
^{e}Only lactosucrose was a substrate in this reaction and the hydrolysis percent was calculated by subtracting the percent transfructosylation from the percent of converted substrates at a specified time point.
^{f}Both lactosucrose and sucrose were substrates in this reaction and the hydrolysis percent was calculated by dividing the fructose quantity at a specified time point by the initial sucrose and lactosucrose quantities multiplied by 100.
^{g}The quantity of released fructose was calculated by taking the fructose quantity at a specified time point and subtracting it from the initial fructose quantity.
^{h}The quantity of released lactose was calculated by taking the lactose quantity at a specified time point and subtracting it from the initial lactose quantity.

These results indicate that both lactosucrose and/or sucrose may have been used as the fructosyl donors by V. natriegens LS2. Focusing more on the lactosucrose V. natriegens LS2 reaction, the concentration of free lactose was higher than that of fructose across the reaction time course, indicating the use of lactose as a fructosyl acceptor and of lactosucrose as a fructosyl donor. The targeted MS-MS analyses of 48 h-G. oxydans LS1, V. natriegens LS2, and B. graminis LS4 reactions where lactosucrose was present by itself revealed the presence of a tetrasaccharide, frustosyl-lactosucrose. This indicates that lactosucrose could be utilized as a fructosyl acceptor by G. oxydans LS1, V. natriegens LS2, and B. graminis LS4.

Selected functionalized supports were investigated for the immobilization of LSs from Gluconobacter oxydans (LS1) and Vibrio natriegens (LS2) by multipoint covalent attachment. The highest immobilization protein yields of 94% and 87% and retained activities of 55% and 98%, respectively, were achieved upon the immobilization of LS1 and LS2 on Relizyme™ EP403/S functionalized with iminodiacetic acid (IDA)-Cu. A greater thermal stabilization of immobilized LSs was achieved after post-immobilization treatments, especially for high pH post-immobilized V. natriegens LS2 with a thermal stability factor of 53. The LS immobilization enhanced the reaction selectivity towards transfructosylation, and the Relizyme™ EP403/IDA-Cu immobilized V. natriegens LS2 led to the highest produced amount of lactosucrose with lactose/sucrose, WP/sucrose, and MP/sucrose (35-95 g/L). The optimal lactose/sucrose ratio and incubation time for the synthesis of lactosucrose with high selectivity by free and immobilized LS2 were identified to respectively be 0.586 and 3.117 h and 0.503 and 3.083 h. These optimal conditions were found to provide 117 g/L with the free V. natriegens LS2 and 101 g/L with the Relizyme™ EP403/S-IDA/Cu immobilized V. natriegens LS2. Lastly, the Relizyme™ EP403/IDA-Cu immobilized V. natriegens LS2 was successfully reused up to 3 consecutive times generating a total of 247 g/L of lactosucrose.

The various pre-immobilization treatments were carried out to modify the glyoxyl agarose support with IDA, IDA-Cu, and TEA, to promote anionic, chelating, and cationic interactions, respectively, and contribute to the multi-covalent attachment of LS on the modified glyoxyl agarose supports. The IDA functionalized glyoxyl agarose support primarily promotes an initial step of physical adsorption through the ionic interactions between the negatively charged IDA groups on the support and positively charged amino acid residues, such as lysine, histidine, and arginine, on the LS's surface. The lysine groups were identified as the most likely amino acid residues that can create reversible imine covalent linkages. The IDA-Cu functionalized glyoxyl agarose support can act as a chelating support and bind to the LS through the chelation of sulfhydryl or amine groups on the LS's surface with the cupric ions on the support. The TEA functionalized glyoxyl agarose support predominantly promotes ionic interactions between the positively charged TEA groups on the support and negatively charged aspartate and glutamate residues on the LS. The completed pre-immobilization treatments can be seen in Table 3 where the functional groups for each treatment were characterized.

TABLE 3
Technical properties of modified supports.

| | Glyoxyl-agarose | Sepabeads ® EC-EP/S R | Relizyme ™M EP403/S R |
|---|---|---|---|
| Average pore diameter | — | 10-20 | 40-60 |
| (nm)^{m} | | | |
| Particle size range (μm)^{m} | 50-150 | 100-300 | 100-300 |
| Minimum oxirane content | — | 100 | 30 |
| (μmol/g wet)^{m} | | | |
| Minimum epoxy | — | 200 | 100 |
| group density | | | |
| (μmol/g dry)^{m} | | | |
| Introduced epoxy group | 214.02 (±2.14)^{b} | 266.34 (±0.25)^{a} | 192.35 (±1.58)^{c} |
| (μmol/g of beads) | | | |
| Bound triethylamine (TEA) | 69.23 (±4.30) | — | — |
| (μmol/g of beads) | | | |
| Bound iminodiacetic acid | 140.99 (±5.97)^{b} | 128.42 (±11.96)^{b} | 180.87 (±14.15)^{a} |
| (IDA) | | | |
| (μmol/g of beads) | | | |
| Bound copper | 144.78 (±1.59)^{c} | 417.29 (±6.99)^{a} | 168.53 (±2.82)^{b} |
| (μmol/g of beads) | | | |

^{m}Data acquired from manufacturer.

FIG. 7 displays the immobilization attempts of G. oxydans LS1 on the aforementioned selected functionalized glyoxyl agarose supports. Unfortunately, it was found that both IDA and TEA functionalized glyoxyl agarose supports failed to significantly immobilize the G. oxydans LS1 even after 72 h of incubation. However, within 5 h of incubation, the IDA-Cu functionalized glyoxyl agarose support was able to immobilize 89.7% (w/w) of the loaded G. oxydans LS1 protein content. This immobilized LS1 protein content was maintained after 24 h of incubation. Therefore, the IDA-Cu functionalization was selected as the pre-immobilization treatment for the immobilization of LSs on the glyoxyl agarose and epoxy activated supports. An incubation time of 5 h was applied for the immobilization of LSs.

The effects of the polymeric properties and functional group density of selected immobilization supports on LS immobilization were evaluated using IDA-Cu functionalization with the glyoxyl agarose support and the two epoxy activated supports Sepabeads© EC-EP and Relizyme™ EP403. As shown in Table 3, these supports differ in their pore size where the glyoxyl agarose support is composed of agarose microspheres with no pores crosslinked for a macroporous structure, while the mesoporous Sepabeads© EC-EP support has the smaller pore size (10-20 nm) in comparison to the macroporous Relizyme™ EP403 support (40-60 nm). For the epoxy activated supports, the oxirane group density of the supports is inversely proportional to their pore size, since supports with a small pore size have a larger surface area. Furthermore, looking at the bound IDA group content, it was found to be similar across the glyoxyl agarose/IDA-Cu and Sepabeads® EC-EP/IDA-Cu supports (128-141 μmol/g of beads) while the Relizyme™ EP403/IDA-Cu was significantly higher (181 μmol/g of beads). However, the bound copper content was different for all three supports as it was much higher for the Sepabeads® EC-EP/IDA-Cu (417 μmol/g of beads) followed by Relizyme™ EP403/IDA-Cu and then the glyoxyl agarose/IDA-Cu and (169 and 145 μmol/g of beads, respectively).

The immobilization results of G. oxydans LS1 and V. natriegens LS2 on the three selected supports functionalized with IDA-Cu are shown in Table 4. All immobilization supports resulted in immobilization protein yields within the range of 86-94% (w/w). These results reveal that the differences in pore diameter and group density of immobilization supports did not affect the immobilization protein yield.

Table 4 indicates that the difference between the investigated immobilized supports laid in the retained activity. The lowest values were obtained with the glyoxyl agarose/IDA-Cu support (26.9-32.2%) followed by the Sepabeads® EC-EP/IDA-Cu support (34.7-42.1%), while the Relizyme™ EP403/IDA-Cu support provided the highest retained activity values of 55% and 98% for G. oxydans LS1 and V. natriegens LS2, respectively.

TABLE 4
The immobilization efficiency of G. oxydans LS1 and V. natriegens
LS2 on each support functionalized with iminodiacetic acid (IDA)-Cu.

| | | | | | Immobilized |
|---|---|---|---|---|---|
| | | | | Specific Activity of | Enzyme Activity |
| | | Immobilization | | Immobilized Enzymes | (μmol/g |
| | | Protein Yield | Retained | (μmol/mg | immobilized |
| Enzyme | Support | (% w/w)^{a} | Activity (%)^{b} | protein*min)^{c} | enzyme*min)^{d} |
| G. oxydans | Glyoxyl Agarose/IDA-Cu | 89.73 (±1.67) | 32.16 (±0.76) | 93.85 (±2.22) | 691.37 (±16.35) |
| LS1 | Sepabeads ®EC-EP/IDA-Cu | 85.95 (±1.31) | 34.71 (±2.51) | 91.62 (±6.62) | 621.77 (±44.94) |
| | Relizyme ™ EP403/IDA-Cu | 94.23 (±0.70) | 55.13 (±2.09) | 205.23 (±7.76) | 1633.55 (±61.80) |
| V. natriegens | Glyoxyl Agarose/IDA-Cu | 93.09 (±0.41) | 26.90 (±0.99) | 21.88 (±0.80) | 200.92 (±7.37) |
| LS2 | Sepabeads ®EC-EP/IDA-Cu | 92.97 (±1.10) | 42.12 (±3.91) | 38.41 (±3.56) | 367.61 (±34.09) |
| | Relizyme ™ EP403/IDA-Cu | 87.42 (±1.35) | 97.48 (±13.77) | 87.09 (±12.30) | 795.13 (±112.32) |

^{a}Immobilization protein yield was calculated by combining the levansucrase protein content in the supernatant and wash and subtracting that from the total levansucrase protein content in the free enzyme solution and then dividing that result by the total levansucrase protein content in the free enzyme multiplied by 100.
^{b}Retained activity was calculated as the ratio of the specific activity of the immobilized enzyme divided by the specific activity of the free enzyme solution multiplied by 100.
^{c}The specific activity of the immobilized levansucrase was calculated by dividing the levansucrase activity by the immobilized levansucrase content.
^{d}The activity of the immobilized levansucrase per gram of support.

These results are due to the occurrence of protein-protein and protein-support interactions at different sites and extents with the three aforementioned supports. The high retained activity obtained upon immobilizing LSs on Relizyme™ EP403/IDA-Cu may be due to the net charge on the surface of the support as a result of the significant modification of the oxirane groups by IDA (181 μmol/g of beads). The IDA group reacts with the oxirane group found in the epoxy activated supports and forms a functional group with two carboxylate anions that will interact with the positively charged amino on the LS, hence promoting physical adsorption. Moreover, the larger pore size of the Relizyme™ EP403/IDA-Cu have aided in the diffusion of substrates and products to and from the active site of LS. On the other hand, the small pore diameter and the high copper density (417 μmol/g of beads) of Sepabeads® EC-EP/IDA-Cu seem to have favored a high extent of protein-protein and protein-support interactions, resulting in LS denaturation and low retention of activity. The use of the glyoxyl agarose/IDA-Cu, which has the lowest copper density (145 μmol/g of beads) and no pores, may have led to LS denaturation and/or active site steric hindrance.

Even though the retained activity value for the Relizyme™ EP403/IDA-Cu immobilized G. oxydans LS1 was lower in comparison to immobilized V. natriegens LS2, immobilized LS1 exhibited higher specific and enzyme activity values. This can be attributed to the high activity of the free LS1. Consequently, the Relizyme™ EP403/IDA-Cu was utilized to immobilize the LSs that were assessed in the biotransformation reactions.

Table 5 summarizes the total, hydrolytic, and transfructosylation activities of free and immobilized LSs with sucrose and lactose/sucrose substrates. The hydrolytic activity was estimated from the fructose content as it is expected to be solely the result of sucrose hydrolysis into glucose and fructose. Conversely, the glucose content provides the total activity corresponding to the hydrolysis of sucrose and the transfructosylation of the fructose unit of sucrose to an acceptor molecule. Therefore, the difference between the glucose and fructose contents is used to estimate the transfructosylation activity.

When sucrose was the sole substrate, all supports immobilizing G. oxydans LS1 provided higher total activities (94.96-323.28 μmol/mg protein min) than the free LS1; while only V. natriegens LS2 immobilized on Relizyme™ EP403/IDA-Cu showed a higher total activity (101.63 μmol/mg protein min) than its corresponding free form. The results also indicate that in the presence of lactose/sucrose substrates, the total activities of immobilized G. oxydans LS1 and V. natriegens LS2 were lower in comparison to their free forms.

By comparing the ratio of total activity with sucrose and sucrose/lactose, the effect of immobilization on the substrate specificity can be inferred. A significant shift of the substrate specificity of G. oxydans LS1 towards sucrose than sucrose/lactose was observed upon immobilization. No change in the substrate specificity of V. natriegens LS2 was observed upon immobilization on Sepabeads® EC-EP/IDA-Cu exhibiting a similar ratio of total activity with sucrose and sucrose/lactose (0.85-0.89). However, other immobilized LS2 exhibited higher substrate specificity towards sucrose than sucrose/lactose. The highest total activity with lactose/sucrose was achieved with G. oxydans LS1 immobilized on Relizyme™ EP403/IDA-Cu (60.36 μmol/mg protein min) and V. natriegens LS2 immobilized on Sepabeads®/IDA-Cu (60.35 μmol/mg protein min). Furthermore, LS2 immobilized on Relizyme™ EP403/IDA-Cu and LS1 immobilized on Sepabeads® EC-EP/IDA-Cu led to more or less similar total activities (52.82-55.51 μmol/mg protein min) in the presence of lactose/sucrose.

TABLE 5
The catalytic efficiency of free and immobilized levansucrases (LS) on each support functionalized with iminodiacetic acid (IDA)-Cu.

| | | | Total | Hydrolytic | Transfructosylation | |
|---|---|---|---|---|---|---|
| | | | Activity | Activity | Activity | Transfructosylation/ |
| | | | (μmol/mg | (μmol/mg | (μmol/mg | Hydrolysis |
| Enzyme | Substrate | Form | protein min)^{a} | protein min)^{b} | protein min)^{c} | Ratio^{d} |
| G. oxydans | Sucrose | Free | 3.60 (±0.01) | 1.04 (±0.01) | 2.57 (±0.01) | 2.46 |
| LS1 | | Glyoxyl Agarose/IDA-Cu | 94.96 (±9.65) | 15.14 (±4.68) | 79.82 (±4.97) | 5.27 |
| | | Sepabeads ®EC-EP/IDA-Cu | 134.82 (±13.87) | 18.73 (±3.00) | 116.09 (±10.87) | 6.20 |
| | | Relizyme ™ EP403/IDA-Cu | 323.28 (±20.19) | 120.81 (±4.32) | 202.47 (±15.87) | 1.68 |
| | Lactose/ | Free | 291.57 (±4.83) | 18.41 (±6.72) | 273.17 (±1.89) | 14.84 |
| | Sucrose | Glyoxyl Agarose /IDA-Cu | 3.19 (±1.59) | 0.01 (±0.001) | 3.19 (±1.59) | 318.50 |
| | | Sepabeads ®EC-EP/IDA-Cu | 55.51 (±9.77) | 19.89 (±6.97) | 35.62 (±2.80) | 1.79 |
| | | Relizyme ™ EP403/IDA-Cu | 60.36 (±9.05) | 0.01 (±0.001) | 60.36 (±9.05) | 6035.80 |
| V. natriegens | Sucrose | Free | 78.02 (±4.24) | 53.25 (±3.10) | 24.77 (±0.11) | 0.47 |
| LS2 | | Glyoxyl Agarose /IDA-Cu | 68.16 (±1.58) | 32.85 (±3.07) | 35.31 (±1.49) | 1.07 |
| | | Sepabeads ®EC-EP/IDA-Cu | 53.62 (±4.63) | 13.82 (±1.38) | 39.80 (±3.25) | 2.88 |
| | | Relizyme ™ EP403/IDA-Cu | 101.63 (±8.86) | 19.32 (±1.47) | 82.31 (±7.39) | 4.26 |
| | Lactose/ | Free | 91.71 (±5.15) | 20.34 (±0.53) | 71.37 (±4.62) | 3.51 |
| | Sucrose | Glyoxyl Agarose /IDA-Cu | 16.37 (±1.38) | 0.01 (±0.001) | 16.37 (±1.38) | 1636.80 |
| | | Sepabeads ®EC-EP/IDA-Cu | 60.35 (±8.47) | 10.01 (±2.02) | 50.35 (±6.45) | 5.03 |
| | | Relizyme ™ EP403/IDA-Cu | 52.82 (±3.39) | 9.80 (±4.90) | 43.02 (±1.51) | 4.39 |

^{a}Total activity was calculated by taking the slope of glucose in mmol/ml · min and multiplying it by the total reaction volume and dividing that by the enzyme content.
^{b}Hydrolytic activity was calculated by taking the slope of fructose in mmol/ml · min and multiplying it by the total reaction volume and dividing that by the enzyme content.
^{c}Transfructosylation activity was calculated by taking the difference between the total activity and hydrolytic activity.
^{d}The ratio of transfructosylation to hydrolysis of each LS.

It can be seen from the results that the immobilized LSs had higher transfructosylation activities than hydrolytic ones, which is desired in order to produce more lactosucrose and FOSs and limit the sucrose hydrolysis side reaction. However, the transfructosylation to hydrolysis ratio was dependent on the type of support and the substrate used. When G. oxydans LS1 was immobilized on glyoxyl agarose/IDA-Cu and Sepabeads® EC-EP/IDA-Cu, it led to high transfructosylation to hydrolysis ratios (5.3-6.2) with sucrose substrate than the corresponding free form of LS1. On the other hand, the immobilized V. natriegens LS2 exhibited higher transfructosylation to hydrolysis ratios than the free LS2 with the highest ratio being achieved with immobilized LS2 on Relizyme™ EP403/IDA-Cu (4.26). The ratio of transfructosylation to hydrolysis was higher when lactose was present for most of immobilized LSs with the sole exception being the immobilized G. oxydans LS1 on Sepabeads® EC-EP/IDA-Cu support showing a decrease in the ratio from 6.20 to 1.79. In the presence of lactose/sucrose, glyoxyl agarose/IDA-Cu immobilized V. natriegens LS2 and Relizyme™ EP403/IDA-Cu immobilized G. oxydans LS1 exhibited the highest transfructosylation to hydrolysis ratios of 1636.8 and 6035.8, respectively. These high ratios may be attributed to the favorable partitioning of lactose, sucrose, and their transfructosylation products at macro/microenvironments of immobilized LSs, promoting their transfructosylation rather than their hydrolysis. The changes in the tridimensional structures of LSs upon immobilization may have also contributed to the modulation of their reaction selectivity. Moreover, as Table 5 shows, both Relizyme™ EP403/IDA-Cu and glyoxyl agarose/IDA-Cu supports have close bound copper content within the range of 144.8-168.5 μmol/g of beads; while the Sepabeads® EC-EP/IDA-Cu support was characterized with a smaller pore size and a higher copper density of 417.3 μmol/g of beads.

Post-immobilization treatments were carried out to better stabilize the LSs immobilized on the functionalized Relizyme™ EP403/IDA-Cu support. One of the post-immobilization treatments encompassed the construction of reversible covalent bonds, particularly Schiff bases, by exposing the complex to high pH conditions for the formation of stronger intramolecular interactions between the LS and the epoxy activated support. Another step included the addition of the polyaminated crosslinker PEI, which is a hydrophilic and cationic polymer at pH 6 allowing for an ionic coating of the immobilized LS by its stronger attachments to negatively charged surfaces, such as the aspartate and glutamate residues on the LS. However, the alkaline conditions required for the promotion of multipoint covalent attachment and the ionic coating by PEI may affect the activity of the immobilized enzyme. FIG. 8 shows that the pH exposure caused a 26% and 58% decrease in activity for immobilized G. oxydans LS1 and V. natriegens LS2, respectively. Consequently, the immobilized G. oxydans LS1 had a higher immobilized enzyme activity value (39.1 μmol/g immobilized enzyme. min) after high pH exposure in comparison to that of the immobilized V. natriegens LS2 (9.7 μmol/g immobilized enzyme. min). Further treatment with the PEI crosslinker caused additional decrease, providing retained activity of 10.5% for immobilized G. oxydans LS1 and 35.5% for immobilized V. natriegens LS2. As a result, both G. oxydans LS1 and V. natriegens LS2 ended up with a similar immobilized enzyme activity of 3.2-3.3 μmol/g immobilized enzyme. min, when the PEI crosslinker was introduced. The results also reveal that contrary to the immobilized V. natriegens LS2, the activity of the immobilized G. oxydans LS1 was more affected by the PEI post-treatment as it had an overall steeper decline in activity.

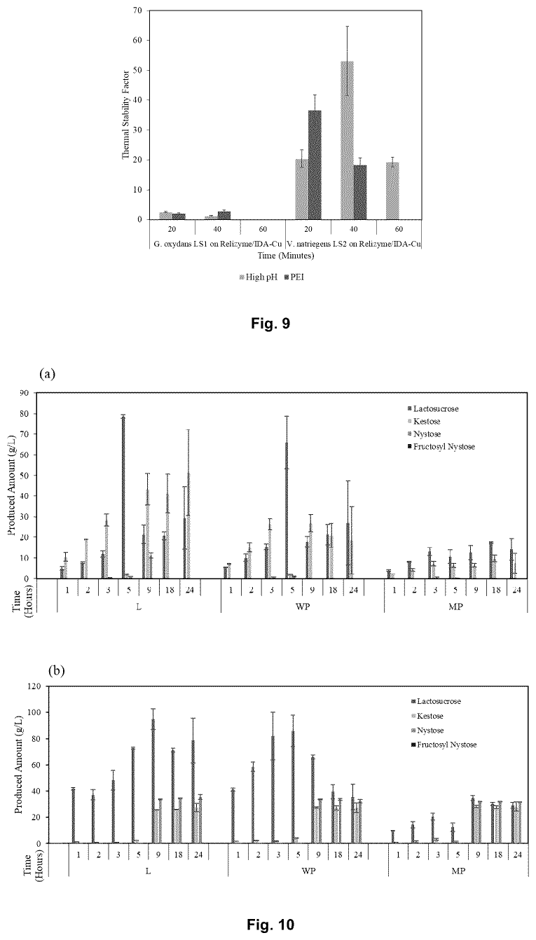

The thermal stability factor was estimated as the ratio of the retained activity of the post-immobilization treated LS (high pH and PEI) to that of the free LS after both were incubated at 50° C. at selected times. FIG. 9 shows the thermal stabilization of Relizyme™ EP403/IDA-Cu immobilized G. oxydans LS1 increase by a factor of 2.5 and 2.7 at 20 and 40 min incubation upon the high-pH and PEI post-immobilization treatments, respectively. However, after 60 min of incubation at 50° C., activity of immobilized G. oxydans LS1 was unfortunately not detectable regardless the type of post-immobilization treatment. On the other hand, the Relizyme™ EP403/IDA-Cu immobilized V. natriegens LS2 that was subjected to high pH post-immobilization treatment showed a more promising thermal stability with a factor of 53.1 after 40 min incubation at 50° C.; while PEI post-immobilized LS2 led to the highest value of 36.6 after 20 min.

The time course for the biotransformation of lactose and dairy by-products in the presence of sucrose by immobilized LSs was investigated. Four end-products, lactosucrose, kestose, nystose, and fructosyl nystose, were quantified using an ion mobility Q-TOF-MS system. The investigated dairy by-products as sources of lactose included WP and MP.

FIG. 10 shows that the Relizyme™ EP403/IDA-Cu immobilized G. oxydans LS1 favored the production of kestose rather than lactosucrose at the beginning of the reaction time course with lactose/sucrose and WP/sucrose. However, after 5 h of reaction time, the amount of produced lactosucrose reached the highest amount of 66-79 g/L, which decreased thereafter and remained at a range of 18-29 g/L at the end of the reaction time course (9-24 h). Contrary to the Relizyme™ EP403/IDA-Cu immobilized G. oxydans LS1, the Relizyme™ EP403/IDA-Cu immobilized V. natriegens LS2 exhibited a higher specificity towards the synthesis of lactosucrose (86-95 g/L), which was most dominant at the 5 h and 9 h reaction time with WP/sucrose and lactose/sucrose, respectively; thereafter, kestose and nystose increased in production and were synthesized within a range of 26-28 and 32-36 g/L, respectively, but lactosucrose was still the most dominant transfructosylation product (35-79 g/L). The decrease in the lactosucrose amount at the end of the reaction time course can be attributed to a shift of the reaction towards the hydrolysis of lactosucrose.

Contrary to lactose/sucrose and WP/sucrose, lactosucrose synthesis was predominant throughout the entire reaction time when MP/sucrose was utilized. However, the total quantity produced from MP/sucrose was smaller than that with other substrates as the initial lactose of MP was limited to 4.8% (w/w). The highest lactosucrose amounts of 18 and 35 g/L were achieved after 18 h with Relizyme™ EP403/IDA-Cu immobilized G. oxydans LS1 and 9 h with Relizyme™ EP403/IDA-Cu immobilized V. natriegens LS2 catalyzed reactions, respectively. With MP/sucrose, the production of lactosucrose by Relizyme™ EP403/IDA-Cu immobilized G. oxydans LS1 remained predominant as the reaction progressed. However, lactosucrose, kestose, and nystose were produced at equal amounts by Relizyme™ EP403/IDA-Cu immobilized V. natriegens LS2 with the MP/sucrose.

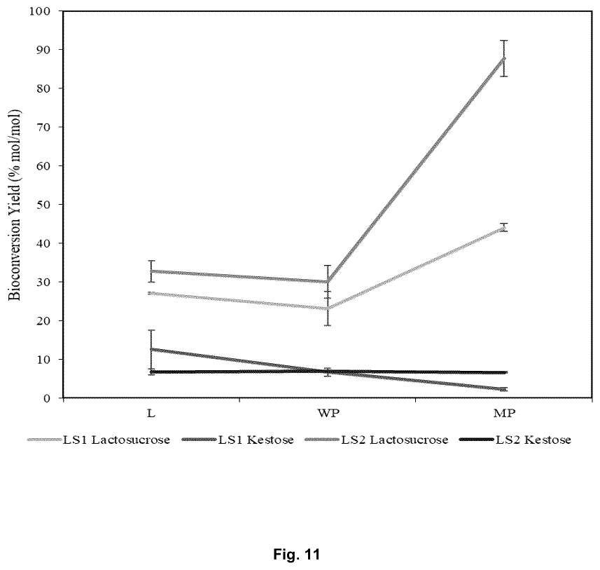

FIG. 11 shows the maximum bioconversion yield of lactosucrose and kestose in all investigated reaction systems. The bioconversion yield of lactosucrose was shown to be higher than that of kestose. The bioconversion yield of kestose was the same for both immobilized LSs when WP/sucrose was utilized (6.7-6.9% mol/mol); however, higher yields were obtained with Relizyme™ EP403/IDA-Cu immobilized G. oxydans LS1 in the presence of lactose/sucrose (12.5% mol/mol) and with Relizyme™ EP403/IDA-Cu immobilized V. natriegens LS2 using MP/sucrose (6.6% mol/mol). However, the highest lactosucrose bioconversion yields were achieved with Relizyme™ EP403/IDA-Cu immobilized V. natriegens LS2 for all three difference lactose sources (30.0-87.8% mol/mol). Consequently, V. natriegens LS2 was determined to be the optimal candidate for lactosucrose production optimization.

The effects of biotransformation parameters were carried out via RSM using WP by-product and sucrose substrates. V. natriegens LS2 was identified as the top candidate for lactosucrose production both in its free and immobilized form on the Relizyme™/IDA-Cu support. The two reaction parameters, the incubation time (3-15 h) and the ratio of lactose to sucrose (0.5-2.0), were varied while the enzyme units (5 U/ml), buffer (50 mM ammonium bicarbonate; pH of 8), and temperature (45° C.) were kept constant. The end-products, lactosucrose, kestose, and nystose, were quantified using the 6560-ion mobility Q-TOF-MS system and their respective amounts (g/L) and yields (% mol/mol) were estimated as the responses (Table 6).

Table 6 shows the analysis of variance (ANOVA) for each response. The F-value indicates the degree of differentiation amongst the various reaction conditions with larger values showing greater statistically significant differences between conditions. The p-value provides a level of statistical confidence in the validity of the test, where small values (ideally less than 0.05) disprove the null hypothesis, which would state that the reaction conditions did not produce a perceivable difference. Contrary to that, the alternative hypothesis would state that the reaction conditions could be perceived as different. For the free form of V. natriegens LS2, the best models that were found to be significant for lactosucrose production were linear for the yield and quadratic for the amount produced.

TABLE 6
Experimental design factors and responses for V. natriegens LS2 in its free form and
immobilized form on Relizyme ™ EP403 functionalized with iminodiacetic acid (IDA)-Cu.

| | | | | | | | | |
|---|---|---|---|---|---|---|---|---|
| | Factor 1 | Factor 2 | Response 1 | Response 2 | Response 3 | Response 4 | Response 5 | Response 6 |
| | Ratio | Time | Lactosucrose | Kestose | Nystose | Lactosucrose Yield | Kestose Yield | Nystose Yield |
| Reaction | Lactose/Sucrose | (Hours) | (g/L) | (g/L) | (g/L) | (% mol/mol) | (% mol/mol) | (% mol/mol) |

Free V. natriegens LS2

| | | | | | | | | |
|---|---|---|---|---|---|---|---|---|
| 1 | 0.72 | 4.76 | 237.17 (±19.54) | 0.00 | 1.05 (±0.06) | 100 (±1.14) | 0.00 | 0.16 (±0.01) |
| 2 | 1.25 | 15.00 | 115.88 (±24.03) | 0.00 | 6.88 (±1.95) | 40.84 (±10.01) | 0.00 | 1.68 (±0.47) |
| 3 | 1.25 | 9.00 | 137.57 (±11.50) | 0.00 | 1.89 (±0.67) | 48.48 (±4.43) | 0.00 | 0.46 (±0.16) |
| 4 | 1.78 | 4.76 | 201.43 (±) | 0.00 | 0.00 | 49.84 (±) | 0.00 | 0.00 |
| 5 | 1.25 | 9.00 | 131.80 (±7.03) | 0.00 | 1.78 (±0.38) | 46.45 (±2.48) | 0.00 | 0.43 (±0.09) |
| 6 | 0.50 | 9.00 | 90.97 (±26.78) | 0.00 | 0.59 (±0.47) | 40.08 (±11.80) | 0.00 | 0.09 (±0.07) |
| 7 | 2.00 | 9.00 | 29.61 (±16.99) | 0.00 | 0.00 | 6.52 (±4.49) | 0.00 | 0.00 |
| 8 | 1.25 | 3.00 | 165.92 (±) | 0.00 | 0.00 | 58.47 (±) | 0.00 | 0.00 |
| 9 | 1.78 | 13.24 | 134.29 (±41.55) | 0.00 | 2.69 (±1.37) | 33.23 (±12.73) | 0.00 | 0.69 (±0.35) |
| 10 | 1.25 | 9.00 | 111.82 (±59.11) | 0.00 | 1.85 (±2.53) | 39.41 (±24.38) | 0.00 | 0.45 (±0.62) |
| 11 | 0.72 | 13.24 | 171.87 (±43.16) | 0.36 (±0.51) | 11.85 (±5.42) | 75.71 (±19.56) | 0.07 (±0.10) | 1.81 (±0.86) |

Immobilized V. natriegens LS2 on Relizyme ™ EP403/IDA-Cu

| | | | | | | | | |
|---|---|---|---|---|---|---|---|---|
| 1 | 0.72 | 4.76 | 148.05 (±35.96) | 4.39 (±0.37) | 19.97 (±0.01) | 65.22 (±19.92) | 0.88 (±0.08) | 3.04 (±0.001) |
| 2 | 1.25 | 15.00 | 27.76 (±4.62) | 2.82 (±0.56) | 15.51 (±0.61) | 9.78 (±1.63) | 0.91 (±0.18) | 3.78 (±0.15) |
| 3 | 1.25 | 9.00 | 26.71 (±6.27) | 1.18 (±0.15) | 14.72 (±0.18) | 9.41 (±2.44) | 0.38 (±0.05) | 3.58 (±0.04) |
| 4 | 1.78 | 4.76 | 103.97 (±11.95) | 2.02 (±0.66) | 17.17 (±0.85) | 25.73 (±2.96) | 0.68 (±0.22) | 4.39 (±0.22) |
| 5 | 1.25 | 9.00 | 32.15 (±9.39) | 1.57 (±0.17) | 14.71 (±0.27) | 11.33 (±4.13) | 0.51 (±0.06) | 3.58 (±0.07) |
| 6 | 0.50 | 9.00 | 141.05 (±31.77) | 8.16 (±1.23) | 18.85 (±1.26) | 62.14 (±15.84) | 1.63 (±0.25) | 2.85 (±0.19) |
| 7 | 2.00 | 9.00 | 58.48 (±19.71) | 5.16 (±0.42) | 20.81 (±0.99) | 12.88 (±5.25) | 2.11 (±0.17) | 6.45 (±0.31) |
| 8 | 1.25 | 3.00 | 74.89 (±5.58) | 0.88 (±0.26) | 13.56 (±0.48) | 26.39 (±1.97) | 0.28 (±0.10) | 3.30 (±0.12) |
| 9 | 1.78 | 13.24 | 41.11 (±5.36) | 1.98 (±0.48) | 17.27 (±0.11) | 10.17 (±1.54) | 0.67 (±0.16) | 4.42 (±0.03) |
| 10 | 1.25 | 9.00 | 49.10 (±8.90) | 2.39 (±0.28) | 16.01 (±0.48) | 17.31 (±3.98) | 0.77 (±0.09) | 3.90 (±0.12) |
| 11 | 0.72 | 13.24 | 72.75 (±22.34) | 7.86 (±0.94) | 23.56 (±0.39) | 32.05 (±12.34) | 1.58 (±0.19) | 3.59 (±0.06) |

With the linear model, it can be seen that both the ratio of lactose to sucrose (F-value of 91.66; p-value of <0.0001) and reaction time (F-value of 16.75; p-value of 0.0011) were significant factors but a higher effect of the ratio was exhibited due to the higher F-value and lower p-value.

For the amount of lactosucrose produced, there was no significant interactive effect between the two factors (F-value of 0.8583; p-value of 0.3725) but the linear effect of reaction time seemed to play a more substantial effect (F-value of 12.05; p-value of 0.0046). The kestose yield and amount produced were equal to zero and thus were not analyzed in relation to the substrate ratio and reaction time parameters. Lastly, the nystose yield and amount produced both fell under a quadratic model and showed no significant interactive effect between the factors (F-value of 0.7364; p-value of 0.4043 and F-value of 3.04; p-value of 0.1016, respectively). Moreover, reaction time appeared to be a more imperative factor for both the nystose yield and amount produced (F-value of 7.7; p-value of 0.0141 and F-value of 7.5; p-value of 0.0153, respectively) (Table 7).

For the Relizyme™ EP403/IDA-Cu immobilized form of V. natriegens LS2, the amount produced and yield of lactosucrose both significantly fit the quadratic model. Both the yield and amount produced displayed no interactive effect between the two factors (F-value of 1.99; p-value of 0.1777 and F-value of 0.1456; p-value of 0.7078, respectively) but both unveiled a more important effect of the ratio of the substrates in the linear and quadratic forms (F-value of 27.5; p-value of <0.0001 and F-value of 27.36; p-value of <0.0001, respectively). The yield and amount produced for kestose and nystose all followed a quadratic model, and only the amount of kestose produced showed an interactive effect between the two factors (F-value of 10.07; p-value of 0.0059) while predictive kestose and nystose yields and amounts models have not shown any interactive effects (F-value of 2.62; p-value of 0.1251, F-value of 0.7979; p-value of 0.3849, and F-value of 1.54; p-value of 0.233, respectively). The ratio of lactose to sucrose seemed to be the significant factor for the kestose yield (F-value of 40.43; p-value of <0.0001) and amount produced (F-value of 108.08; p-value of <0.0001) in addition to the nystose yield (F-value of 11.9; p-value of 0.0033) and amount produced (F-value of 24.32; p-value of 0.0002) (Table 7).

TABLE 7
Analysis of variance (ANOVA) for V. natriegens LS2 in its free form and immobilized
form on Relizyme ™ EP403 functionalized with iminodiacetic acid (IDA)-Cu.

| | | Lactosucrose | | | | Kestose | | | | Nystose | | | |
|---|---|---|---|---|---|---|---|---|---|---|---|---|---|
| | | Yield | | Lactosucrose | | Yield | | Kestose | | Yield | | Nystose | |
| | | (% mol/mol) | | (g/L) | | (% mol/mol) | | (g/L) | | (% mol/mol) | | (g/L) | |
| Form | | F | p-value | F | p-value | F | p-value | F | p-value | F | p-value | F | p-value |
| Free | Model | 54.2 | <0.0001 | 6.39 | 0.0041 | — | — | — | — | 15.9 | <0.0001 | 13.8 | <0.0001 |
| V. natriegens | A- Ratio | 91.66 | <0.0001 | 6.71 | 0.0236 | — | — | — | — | 2.17 | 0.1618 | 5.9 | 0.0282 |
| LS2 | Lactose/ | | | | | | | | | | | | |
| | Sucrose | | | | | | | | | | | | |
| | B- Time (Hours) | 16.75 | 0.0011 | 15.89 | 0.0018 | — | — | — | — | 59.21 | <0.0001 | 51.76 | <0.0001 |
| | AB | — | — | 0.8583 | 0.3725 | — | — | — | — | 0.7364 | 0.4043 | 3.04 | 0.1016 |
| | A² | — | — | 4.38 | 0.0583 | — | — | — | — | 5.93 | 0.0278 | 2.4 | 0.1422 |
| | B² | — | — | 12.05 | 0.0046 | — | — | — | — | 7.7 | 0.0141 | 7.5 | 0.0153 |
| | Lack of Fit | 1.22 | 0.3741 | 4.03 | 0.052 | 1.43 | 0.2765 | 1.43 | 0.2765 | 1.02 | 0.4171 | 2.95 | 0.757 |
| Immobilized | Model | 20.23 | <0.0001 | 12.94 | <0.0001 | 11.22 | <0.0001 | 41.41 | <0.0001 | 19.44 | <0.0001 | 6.42 | 0.0019 |
| V. natriegens | A- Ratio | 54.97 | <0.0001 | 17.44 | 0.0007 | 0.4911 | 0.4935 | 63.68 | <0.0001 | 77.42 | <0.0001 | 2.53 | 0.131 |
| LS2 on | Lactose/ | | | | | | | | | | | | |
| Relizyme ™ | Sucrose | | | | | | | | | | | | |
| EP403/ | B- Time (Hours) | 16.7 | 0.0009 | 19.75 | 0.0004 | 6.32 | 0.023 | 15.54 | 0.0012 | 2.29 | 0.15 | 2.64 | 0.124 |
| IDA-Cu | AB | 1.99 | 0.1777 | 0.1456 | 0.7078 | 2.62 | 0.1251 | 10.07 | 0.0059 | 0.7979 | 0.3849 | 1.54 | 0.233 |
| | A² | 27.5 | <0.0001 | 27.36 | <0.0001 | 40.43 | <0.0001 | 108.08 | <0.0001 | 11.9 | 0.0033 | 24.32 | 0.0002 |
| | B² | 2.4 | 0.1407 | 2.89 | 0.1087 | 0.2657 | 0.6132 | 0.007 | 0.9342 | 1.17 | 0.2958 | 0.2116 | 0.6517 |
| | Lack of Fit | 1.35 | 0.3006 | 2.03 | 0.1589 | 11.49 | 0.0006 | 3.35 | 0.0526 | 27.65 | <0.0001 | 37.23 | <0.0001 |

The free form of V. natriegens LS2 has a lactosucrose yield that fits a linear model while the lactosucrose amount produced and nystose yield and amount produced all fit a quadratic model. The immobilized V. natriegens LS2 on Relizyme ™ EP403/IDA-Cu lactosucrose, kestose, and nystose yields and amounts produced all fit a quadratic model.

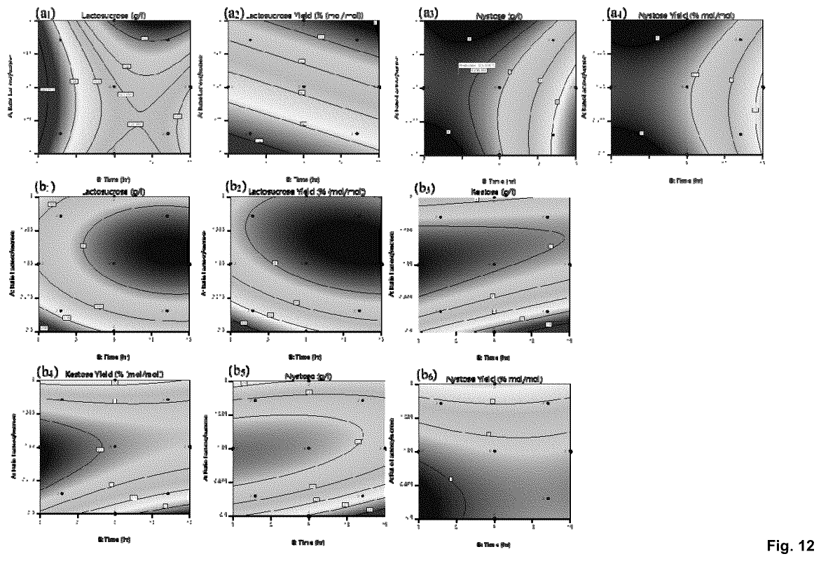

FIG. 12 shows the contour plots of the predictive models. FIG. 12 a₁ reveals that the substrate ratio does not affect the lactosucrose production by free V. natriegens LS2 at the initial stage of the reaction, but its effect became more prominent at the middle and the end of the reaction time course.

A higher amount of produced lactosucrose by free V. natriegens LS2 was achieved at a shorter reaction time, regardless of the substrate ratio. FIG. 12 a₂ displays the lactosucrose bioconversion yield and shows that both a lower substrate ratio and reaction time were needed for a higher lactosucrose bioconversion yield. The results from FIG. 12 a₃ indicate that the longer is the reaction time, the higher is the amount of nystose that is produced in the free V. natriegens LS2 reaction system. FIG. 12 a₃ also demonstrates that a lower substrate ratio is needed to produce more nystose by the free V. natriegens LS2. Therefore, both the reaction time and the substrate ratio influenced the free V. natriegens LS2 reaction system, but the reaction time had a narrower range making it more critical. The bioconversion yield for nystose shown in FIG. 12 a₄ gave similar results as presented with the amount of produced nystose in FIG. 12 a₃; therefore, a higher nystose bioconversion yield with free V. natriegens LS2 requires a longer reaction time and lower substrate ratio.

In the Relizyme™ EP403/IDA-Cu immobilized V. natriegens LS2 biocatalytic system, both lactosucrose bioconversion yield and amount exhibited similar predictive patterns requiring lower substrate ratios and shorter incubation times for maximal lactosucrose synthesis as can be seen in FIG. 12 b₁ and b₂. The predictive models of kestose bioconversion yield and amount produced (FIG. 12 b₃ and b₄) also followed similar patterns of lower substrate ratios and longer reaction times. A similar predictive pattern to that of kestose is shown by the amount of nystose produced in FIG. 12 4.6 b₅ requiring a lower substrate ratio and longer reaction time. However, the nystose bioconversion yield displayed in FIG. 12 b₆ indicates that the substrate ratio was the vital factor, as previously confirmed with the ANOVA table (Table 7), and appeared to require a higher substrate ratio for a higher nystose bioconversion yield.

The selected biotransformation parameters to maximize the selectivity of free and Relizyme™ EP403/IDA-Cu immobilized V. natriegens LS2 towards lactosucrose synthesis were determined from the predictive models. Table 8 summarizes the identified conditions, predictive responses as well as the experimental ones. The experimental lactosucrose bioconversion yield and produced amount fitted the confidence interval for both free and Relizyme™ EP403/IDA-Cu immobilized V. natriegens LS2, revealing the significance of the developed predictive models. Both a shorter reaction time and lower lactose/sucrose ratio are needed to achieve a high selective production of lactosucrose. The free V. natriegens LS2 was able to synthesize 117 g/L of lactosucrose while the Relizyme™ EP403/IDA-Cu immobilized V. natriegens LS2 produced 101 g/L of lactosucrose.

TABLE 8
Responses of the optimal conditions for lactosucrose production with V. natriegens LS2 in its free
form and immobilized form on Relizyme ™ EP403 functionalized with iminodiacetic acid (IDA)-Cu.

| | | | | | | Confidence | Confidence |
|---|---|---|---|---|---|---|---|
| | | | | Lactosucrose | | Interval for | Interval for |
| | | Ratio | Time | Yield | Lactosucrose | Lactosucrose | Lactosucrose |
| | Reaction | Lactose/Sucrose | (Hours) | (% mol/mol) | (g/L) | Yield (% mol/mol) | (g/L) |
| Predicted | Free | 0.586 | 3.117 | 87.096 | 176.067 | 59.5-94.68 | 46.95-185.095 |
| Responses | Immobilized | 0.503 | 3.083 | 51.91 | 100.257 | 43.275-72.415 | 77.71-153.718 |
| Experimental | Free | 0.586 | 3.117 | 51.373 (±9.165) | 116.615 (±20.804) | — | — |
| Responses | Immobilized | 0.503 | 3.083 | 44.394 (±6.962) | 100.773 (±15.803) | — | — |

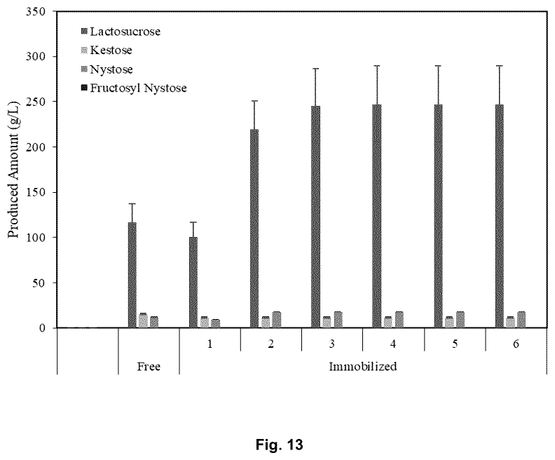

One of the major advantageous features of immobilizing enzymes is the reusability, which is dependent on several factors such as the enzyme's thermal stability and stability from being leached from the immobilization support. The Relizyme™ EP403/IDA-Cu immobilized V. natriegens LS2 was reused up to 5 times but after only 3 times, the amount of produced lactosucrose reached a plateau as seen in FIG. 13. The initial biotransformation reaction provided 101 g/L of lactosucrose while the first time the complex was reused provided an even higher amount with 119 g/L of lactosucrose. Afterwards, the second time the immobilized LS2 was reused offered 25 g/L of lactosucrose and the third and last only gave 2 g/L of lactosucrose. Therefore, the final combined amount of lactosucrose produced was 247 g/L after 4 consecutive cycles before there was no more lactosucrose synthesis.

Out of all the tested immobilization supports and functionalizations, the Relizyme™ EP403 support functionalized with IDA-Cu led to the highest immobilization protein yields and retained activities for G. oxydans LS1 and V. natriegens LS2. Furthermore, the post-immobilization treatments were found to decrease the activities with the Relizyme™ EP403/IDA-Cu immobilized G. oxydans LS1 being less impacted by high pH incubation than the Relizyme™ EP403/IDA-Cu immobilized V. natriegens LS2. However, both LSs did end up reaching a similar relatively low immobilized enzyme activity value with the addition of the PEI crosslinker. Nonetheless, the aforementioned post-immobilization treatments appeared to have provided further thermal stabilization of LSs, particularly the Relizyme™ EP403/IDA-Cu immobilized V. natriegens LS2 treated with high pH. Similar to their free forms, the immobilized LSs favored the transfructosylation reactions that occurred at a rapid rate. The Relizyme™ EP403/IDA-Cu immobilized V. natriegens LS2 was able to catalyze the production of the highest lactosucrose amount with all substrate combinations lactose/sucrose, WP/sucrose, and MP/sucrose. Therefore, V. natriegens LS2 was the LS of choice for the optimization of the lactosucrose synthesis. Predictive models were developed for the biotransformation WP/sucrose systems, catalyzed by free and immobilized LS2, and revealed the significance of reaction time and substrate ratio in modulating the reaction selectivity. Finally, the Relizyme™ EP403/IDA-Cu immobilized V. natriegens LS2 afforded the additional advantage of reusability where it was successfully reused.

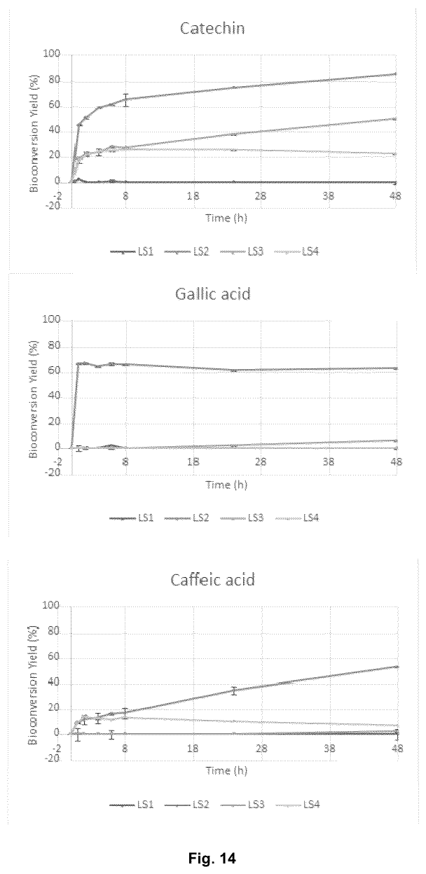

As further encompassed is the additional transfructosylation of phenolic compounds, as present e.g. in chocolate. As shown in FIG. 14, LS2 proved to be the most promising enzyme for the transfructosylation of phenolic compounds present in chocolate, including catechin, gallic acid and caffeic acid reaching a bioconversion yield of about 85%, 60% and 50% respectively. LS3 and LS4 also had a significant bioconversion yield of 50% and 20%, respectively, with catechin.

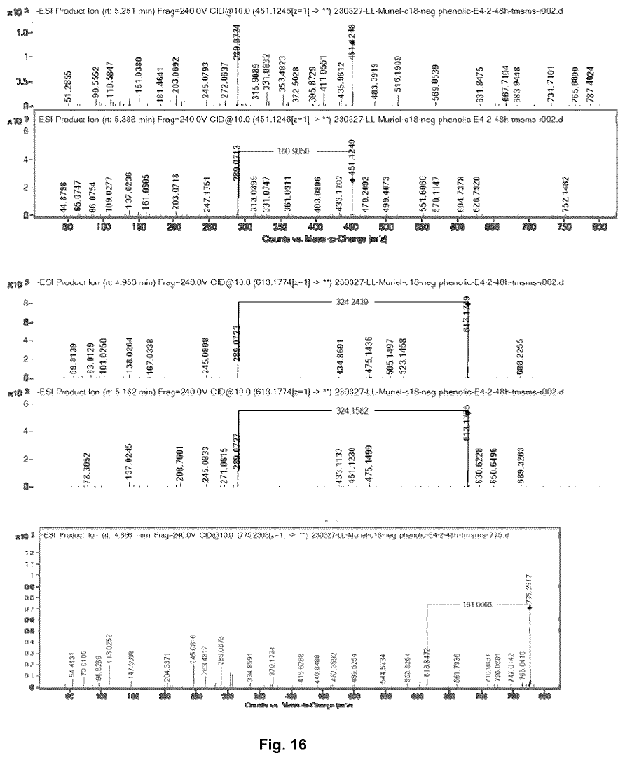

The end-product profile LC-MS characterization confirmed the successful transfructosylation of phenolic compounds catechin, gallic acid, caffeic acid and epicatechin. The LCMS analysis demonstrated that the phenolic compounds could acquire more than one fructosyl group. The MS/MS spectra of fructosylated catechin products and their corresponding possible structures obtained from reaction with LS3 are shown in FIG. 16.

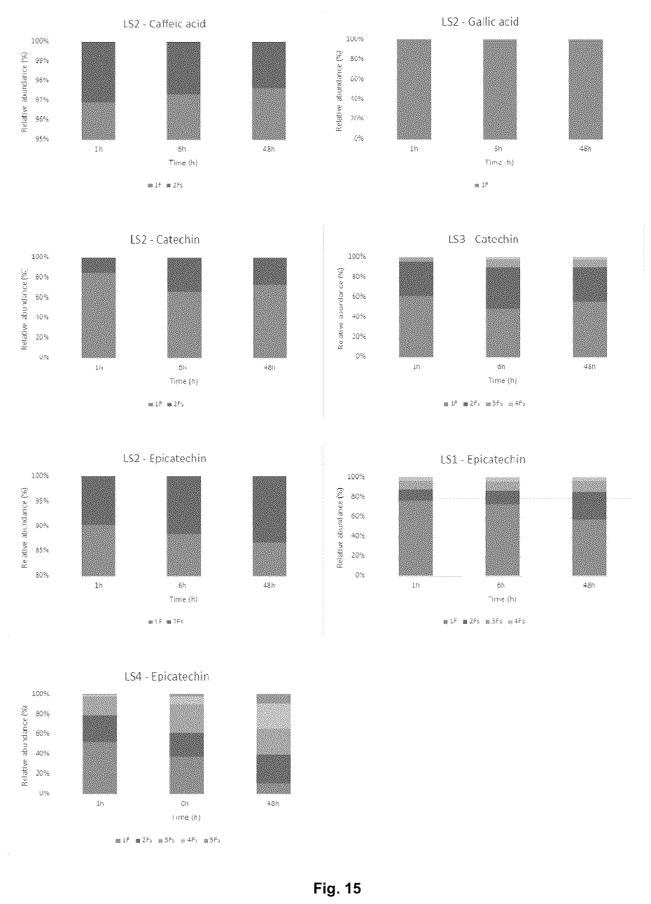

The number of fructosyl groups acquired depends on the phenolic acceptors as well as the source of the enzyme. For e.g., in FIG. 15, with epicatechin, LS2 could acquire two fructosyl groups, LS1 four fructosyl groups and LS4 up to five fructosyl groups. The relative abundance of each transfructosylated phenolic compounds also vary with reaction time. In general, as shown in Table 9, LS2 was proven to be the most promising enzyme for the transfructosylation of selected phenolic compounds.

TABLE 9
Summary of number of fructosyl groups acquired
by transfructosylated compounds

Number of fructosyl groups acquired

| | LS1 | LS2 | LS3 | LS4 |
|---|---|---|---|---|
| Catechin | | 1, 2 | 1, 2, 3, 4 | |
| Gallic acid | | 1 | | |
| Epicatechin | 1, 2, 3, 4 | 1, 2 | | 1, 2, 3, 4, 5 |
| Caffeic acid | | 1, 2 | | |

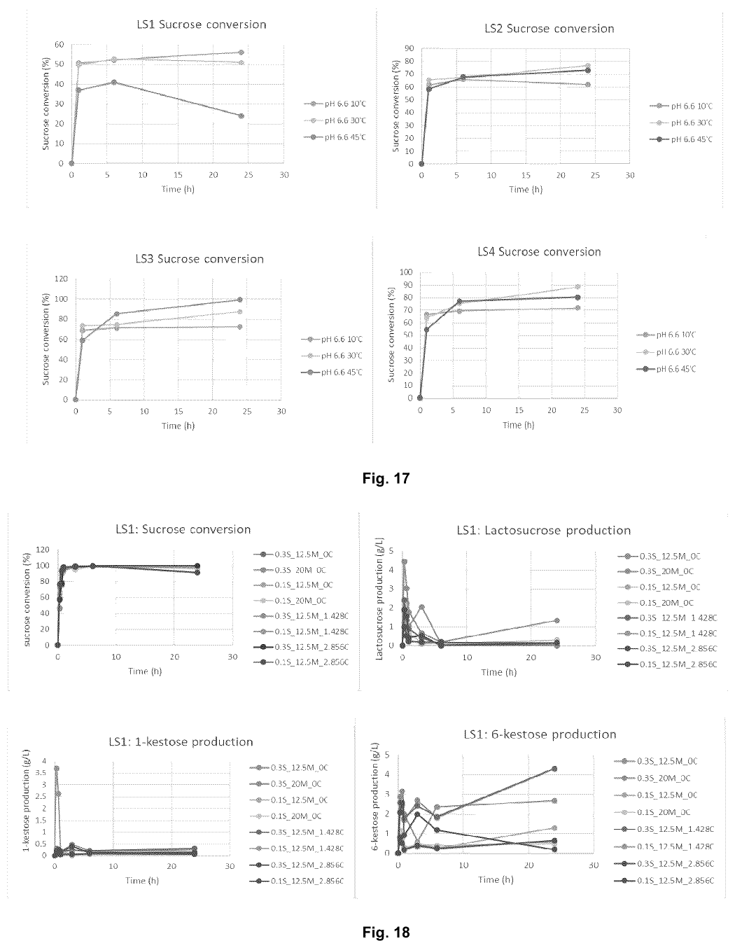

FIG. 17 shows that the sucrose conversion yield was not significantly affected by temperature for LS2, LS3 and LS4 at pH 6.6 (of milk). A high sucrose conversion range of 60-80% for LS2, 70-100% for LS3 and 70-90% for LS4 were recorded. LS1 had a lower range of 25-60%.

In terms of lactosucrose production, LS2 led to the most favourable results with overall higher production yield, even at different temperatures, after only 6 hr of reaction. Given 10° C. is very close to the temperatures used in dairy products processing, LS2 is a very promising enzyme.

Although LS3 had low lactosucrose production, various fructooligosaccharides, namely, 1-kestose, 6G-kestose and nystose, were produced in quite significant amounts.

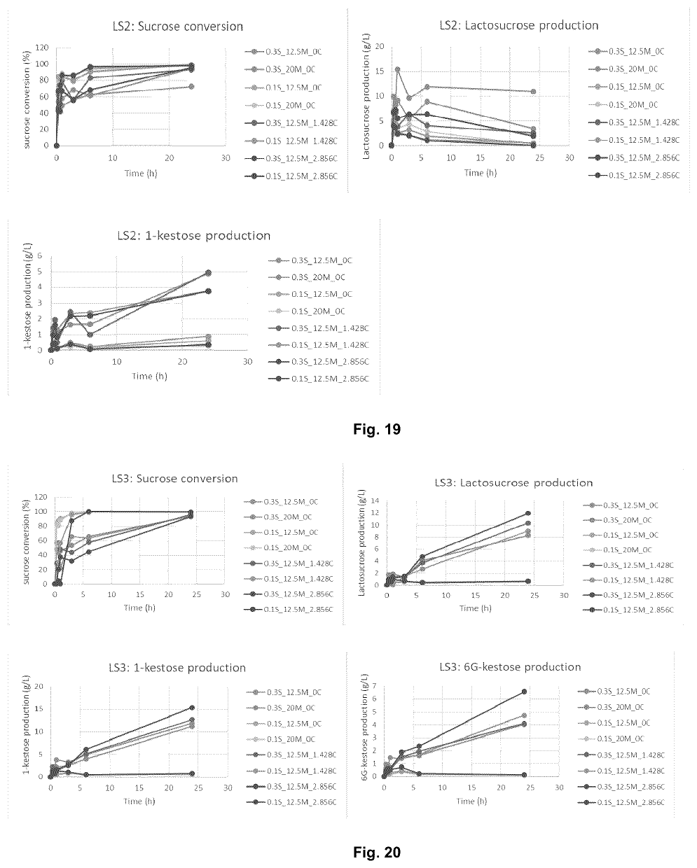

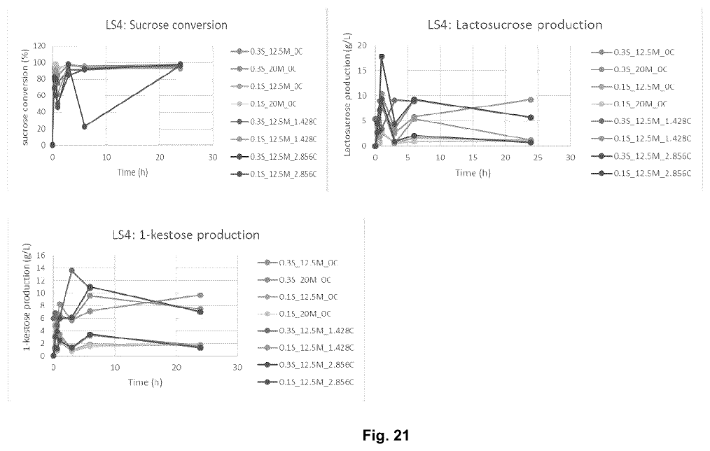

FIGS. 18-20 show that in general milk and cocoa powder do not affect LS-catalysed reactions.

After 24 hr of reaction, almost all sucrose was successfully converted with all four enzymes. In terms of lactosucrose production, LS2, LS3 and LS4 recorded significant yields. It can be clearly seen in FIG. 19 that all reactions carried at 0.3 M sucrose concentration had similar lactosucrose production, hence demonstrating that milk and cocoa powder did not affect the LS3-catalyzed reactions.

1-kestose was produced mainly by LS3 and LS4, reaching around 10 g/L yield after 24 hr of reaction. Other fructooligosaccharides were detected, namely 6-kestose with LS1 and 6G-kestose with LS3. The highest yield recorded was around 4.5 g/L of 6-kestose with LS1, and around 6.5 g/L of 6G-kestose with LS3.

Accordingly, it is provided a process for producing lactosucrose from a source of lactose comprising contacting the dairy starting material (containing sucrose) with a levansucrase which allows lowering sucrose content in dairy products while producing lactosucrose which can also increase bioavailability of phenolic compounds.

### Example I

### Expression and Purification of Selected Levansucrases and Characterization

Sucrose, D-(−)-fructose, D-(+)-glucose, D-(+)-galactose, α-lactose, myo-inositol, 3,5-dinitrosalicylic acid (DNS), potassium sodium tartrate (KNaC₄H₄O₆), yeast extract, carbenicillin disodium salt, lysozyme from chicken egg white, DNase I, imidazole, C₂H₃NaO₂, C₂H₇NO₂, NH₄HCO₃, NaOH solution, and dextran standards (12-670 kDa) were obtained from Sigma-Aldrich (Oakville, ON). Fructooligosaccharide (FOS) standards (i.e. 1-kestose, nystose, and 1^{F}-fructofuranosylnystose) and lactosucrose were supplied by FUJIFILM Wako Chemicals U.S.A. Corporation (Richmond, VA). Bradford reagent concentrate and sodium dodecyl sulfate polyacrylamide gel electrophoresis (SDS-PAGE) low range standards were purchased from Bio-Rad (Missasauga, ON). K₂HPO₄, KH₂PO₄, NaOH (Pellets/Certifies ACS), acetonitrile (ACN) HPLC grade, water optima LC/MS grade, bovine serum albumin (BSA), tryptone, NaCl, β-D-isothiogalactopyranoside (IPTG), PIPES, glycerol, tris hydrochloride, and tris-glycine-SDS 10× solution were provided by Fisher Scientific (Fair Lawn, NJ). Terrific broth (TB) and lysogeny broth (LB) agar powder were acquired from Bio Basic (Markham, ON). E. coli BL21(DE3) plysE strains were supplied by Invitrogen. WP (96.02% total solids made up of 11.55% protein, 7.57% ash, and a minimum of 76.09% lactose by weight) and MP (5.65% total solids made up of 0.25% protein, 0.60% ash, and a minimum of 4.80% lactose by weight) were obtained from a local dairy cooperative.

The genes corresponding to LS1 from G. oxydans (strain 621H), LS2 from V. natriegens NBRC 15636, LS3 from N. aromaticivorans, and LS4 from B. graminis C4D1M were transformed into E. coli BL21-CodonPlus (DE3)-RIPL (Invitrogen) and precultured into LB media also containing carbenicillin (1 μl/ml). The preculture was incubated in an orbital shaker for 8-10 h at 37° C. under 250 rpm. The preculture (2% v/v) was added to the TB with carbenicillin (1 μl/ml), which was then incubated at 37° C. under 250 rpm. When growth was achieved an optical density of 1.2-1.6 at 600 nm (DU 800 UV/Visible Spectrophotometer, Beckman), the enzyme expression was induced using IPTG (1 mM). Growth of the culture was continued at room temperature for 18 h under 250 rpm. The cells were then collected by centrifugation at 4° C. under 8,000 rpm and then stored at −80° C. The recovered pellets were resuspended in the sonication buffer (50 mM PIPES, 300 mM NaCl, and 10% glycerol; pH of 7.2; 4 ml/g). Lysozyme (4 mg/g) and DNase (4 μl/g) were added the suspensions, and the mixtures were incubated at 18° C. under 50 rpm for 1 h. The suspensions were thereafter sonicated with a microtip (Misonix Ultrasonic Liquid Processor S-4000) for six cycles (10 seconds on and 60 seconds off at 15 kHz) in an ice bath. The supernatants containing the enzymes were recovered by centrifugation at 4° C. under 14,000 RPM for 1 h and then dialyzed against potassium phosphate buffer (5 mM; pH of 6) using a membrane with a molecular weight cut-off of 6-8 kDa. The LSs were purified via immobilized metal affinity chromatography on a HisTrap™ FF column (5 ml, GE Healthcare). After loading, the column was subsequently washed with sonication buffer (9 volumes), wash buffer (50 mM PIPES, 300 mM NaCl, and 10% glycerol; pH of 6.4; 9 volumes), 5 mM imidazole-wash buffer (9 volumes), and 10 mM imidazole-wash buffer (9 volumes). The LS enzyme was then eluted with 100 mM and 200 mM imidazole-wash buffers (2 volumes), respectively. Finally, the purity of the LSs was confirmed upon the SDS-PAGE electrophoresis analysis at 120V using 15% SDS polyacrylamide gels and a 10× diluted Tris/Glycine/SDS buffer.

One unit of the total LS activity was expressed as a quantity of biocatalyst that released 1 μmol of reducing sugars, glucose and fructose, from sucrose per min. The total LS activity includes both hydrolytic and transfructosylation activity. The LS hydrolytic unit was expressed as the amount of biocatalyst that generated 1 μmol of the fructose per min, while one transfructosylation unit was expressed as the amount of biocatalyst that produced 1 μmol of glucose, due to the transfer of fructose, per min. Subtracting the total amount of fructose from that of glucose offers the amount of glucose resulting from transferring fructose. All assays were run in duplicates or triplicates.

The total LS activity assay was initiated by mixing purified LS solution with a sucrose substrate solution (1.8M) in potassium phosphate buffer (50 mM; pH of 6) at a ratio of 1:1 (v/v). After 20 min of incubation at 30° C. under 50 rpm, the reaction was terminated by adding the DNS reagent [1% (w/v) 3,5-DNS and 1.6% (w/v) NaOH] at a ratio of 1:1.5 (v/v). The mixtures were then placed in boiling water for 5 min and thereafter, the potassium sodium tartrate (50% w/v) was added to stabilize the colour at a ratio of 1:0.2 (v/v). The absorbance was measured at 540 nm and the reducing sugar concentration was quantified using a standard curve constructed from glucose (0-20 mM).

For the hydrolytic and transfructosylation activities, purified LS solution was added to a sucrose solution to yield a final concentration of 0.9 M in potassium phosphate buffer (50 mM, pH 6.0). Glucose, fructose, and sucrose were quantified by high-pressure anion-exchange chromatography (HPAEC) using a Dionex ICS-3000 system equipped with a pulsed amperometric detector (PAD) and a CarboPac PA20 column (3×150 nm). The components of reaction mixtures were eluted with an isocratic mobile phase made of 20 mM sodium hydroxide at a flow rate 0.4 mL/min and 32° C.

To produce lactosucrose, the enzymatic biotransformation reactions were carried out using sucrose and lactose or lactose containing dairy by-products as substrates (WP and MP). Sucrose and lactose solutions were prepared at a ratio of 1:2 to reach a final concentration of 0.9M:0.45M. To initiate the biotransformation reactions, purified LS (5 U/ml) was added to the substrate solution. The biotransformation reactions were carried out at the optimal conditions for each LS, corresponding to 30° C./pH 4 (50 mM sodium acetate buffer) for G. oxydans LS1, 45° C./pH 8 (50 mM tris-HCl buffer) for V. natriegens LS2, 45° C./pH 6 (50 mM potassium phosphate buffer) for N. aromaticivorans for LS3, and 30° C./pH 4 (50 mM sodium acetate buffer) for B. graminis LS4. All reactions were done in duplicates under 50 rpm. A blank where no enzyme was added was run in parallel for each LS. The biotransformation reactions were carried out over a time course of 48 h where aliquots were taken, placed in boiling water for 3 min to stop the reaction, and then stored at −20° C. until further analysis.

The reaction selectivity was assessed in the presence of sucrose alone or with lactose or lactose-containing dairy by-products (WP and MP) as substrates using the four selected LSs. After the enzymatic biotransformation reactions, the remaining sucrose and lactose as well as the released galactose, glucose, and fructose were quantified by HPAEC using a Dionex ICS-3000 system equipped with a PAD and a CarboPac PA20 column as described above. The hydrolysis extent of sucrose was quantified from the concentration of released fructose and taken as a percentage of the initial sucrose concentration, while that of lactose was expressed as the galactose concentration as a percentage of the initial lactose concentration. The extent of sucrose transfructosylation was based on the difference between the concentrations of fructose and glucose as a percentage of the initial sucrose concentration, whereas the extent of lactose transfructosylation was calculated from the difference between lactose and galactose concentrations overtime as a percentage of the initial lactose concentration.

To assess the donor-acceptor specificity of LSs towards lactosucrose, biotransformation reactions were carried out in the presence of lactosucrose alone (0.45M) and lactosucrose/sucrose (0.45M/0.45M) substrates. The biotransformation reactions were initiated by the addition of G. oxydans LS1 (30° C./pH 4), V. natriegens LS2 (45° C./pH 8), N. aromaticivorans for LS3 (45° C./pH 6), or B. graminis LS4 (30° C./pH 4) at a concentration of 5 U/ml. Each reaction was carried out in duplicates and a reactions blank, without a LS enzyme, ran in parallel. At selected time intervals, aliquots were taken, placed in boiling water for 3 min to stop the reaction, and stored at −20° C. until end-product characterization.

The end-products of the biotransformation reactions were analyzed using an Agilent 1290 II liquid chromatography system coupled to an Agilent 6560-ion mobility Q-TOF-MS. The analytes were separated with an InfinityLab Poroshell 120 HILIC-Z column (2.1×100 mm, 2.7 μm). Mobile phase A was LC-MS grade water with 0.3% NH₄OH and mobile phase B was ACN with 0.3% NH₄OH. Flow rate was set at 0.4 ml/min with a column temperature of 35° C. The constructed gradient started off with 85% B (0.0 to 0.5 min) that had a linear decrease to 30% B (0.5 to 9.0 min) where it was held (9.0 to 13.0 min) and then increased to 85% B (13.0 to 15.0 min), followed by a 3 min post-run. The mass spectrometer was equipped with a Dual AJS ESI ion source operating in negative ionization mode. MS conditions for ESI were as follows: drying gas temperature of 150° C. and flow rate of 11 L/min, sheath gas temperature of 350° C. and flow rate of 12 L/min, pressure on the nebulizer of 30 psig, capillary voltage of 4000V, fragmentor voltage of 200V, skimmer voltage of 30V, and nozzle voltage of 2000V. Full scan MS data was recorded at mass-to-charge ratios (m/z) from 80 to 1100 at a scan rate of 2 spectra/s and was collected at both centroid and profile mode. Reference ions (m/z at 112.985587 and 1033.988109 for ESI-) were used for automatic mass recalibration of each acquired spectrum. The quantification was performed using Quantitative Analysis 10.0 from Agilent MassHunter Workstation Software. The samples were prepared by dilution in 50:50 ACN:Water (v/v) with the addition of myo-inositol (5-30 ppm) to serve as an internal standard.

Furthermore, the production of oligomers and levans was assessed by high-pressure size-exclusion chromatography (HPSEC) utilizing a Waters HPLC system equipped with a 1525 binary pump, refractometer 2489 detector, and TSKgel G5000PWXL-CP column (7.8 mm×30 cm, 5 μm). An isocratic mobile phase made up of 200 mM NaCl was set at a flow rate of 0.5 mL/min and the molecular weight distribution was determined using a standard curve constructed with dextrans of different molecular weights (12, 50, 270, and 670 kDa).

The protein content of the LS solution was quantified using the Bradford protein assay with a BSA standard (1-20 μg/ml). The specific enzyme activity was expressed as the unit of biocatalyst in μmol of reducing sugar per min per mg of protein.

### Example II

### Immobilization of Levansucrase

4-morpholinepropanesulfonic acid (MOPS), epichlorohydrin, H₂SO₄, NaIO₄, IDA, CuSO₄, NaH₂PO₄, Na₂HPO₄, and polyethylenimine (PEI) were obtained from Sigma-Aldrich (Oakville, ON). FOS standards (i.e. 1-kestose, nystose, and 1^{F}-fructofuranosylnystose) and lactosucrose were supplied by FUJIFILM Wako Chemicals U.S.A. Corporation (Richmond, VA). Sodium dodecyl sulfate polyacrylamide gel electrophoresis (SDS-PAGE) low range standards were purchased from Bio-Rad (Mississauga, ON). KH₂PO₄, K₂HPO₄, NaOH (Pellets/Certifies ACS), acetonitrile (ACN) HPLC grade, water optima LC/MS grade, bovine serum albumin (BSA), tryptone, NaCl, β-D-isothiogalactopyranoside (IPTG), PIPES, glycerol, tris-glycine-SDS 10× solution, NaBH₄, acetone, triethylamine (TEA), and Pierce™ Coomassie Plus (Bradford) assay kit were provided by Fisher Scientific (Fair Lawn, NJ). Terrific broth (TB) and lysogeny broth (LB) agar powder were acquired from Bio Basic (Markham, ON). 10% BCL agarose bead standard (50-150 μm) was purchased from Agarose Bead Technologies (Doral, FL). Escherichia coli BL21(DE3) plysE strains were supplied by Invitrogen. Sepabeads® EC-EP/S R and Relizyme™ EP403/S R were obtained from Resindion® (Binasco, Lombardy, Italy). WP (96.02% total solids made up of 11.55% protein, 7.57% ash, and a minimum of 76.09% lactose by weight) and MP (5.65% total solids made up of 0.25% protein, 0.60% ash, and a minimum of 4.80% lactose by weight) were obtained from a local dairy cooperative.

The protocol for the preparation of glyoxyl-based supports followed the method described by Hill et al. (2016, Journal of Chemical Technology & Biotechnology, 91(9): 2440-2448). A round bottom flask was placed on ice where NaOH (0.656M; 440 ml), NaBH₄ (2 g), acetone (160 ml), and washed 10% BCL Agarose (100 g) were added. Epichlorohydrin (110 ml) was then introduced to the mixture and stirred overnight at 25° C. The support was finally washed with deionized water (10 volumes). The epoxy-activated agarose (10% w/v) was then suspended in the acetone:water solution (1:1 v/v). TEA was slowly added to reach a final concentration of 0.1M and if needed, the pH was adjusted to reach a value within the range of 12.5-13. The mixture was stirred for 48 h and washed afterwards with distilled water (10 volumes). The remaining hydroxyl groups were oxidized with NaIO₄ (0.01M; 5% v/v) for 90 min and then the support was recovered by filtration and washed with distilled water (10 volumes).

The epoxy-activated agarose (10% w/v) was suspended in an IDA solution (0.5M; pH of 11) for 36 h at 25° C. The IDA-functionalized glyoxyl agarose support was then recovered by filtration and washed with distilled water (10 volumes). The remaining hydroxyl groups were oxidized with NaIO₄ (0.01M; 5% v/v) for 90 min and then the mixture was filtered to recover the modified support, which was washed with distilled water (10 volumes).

The support was functionalized with the additional steps of suspending in CuSO₄ (30 mg/ml) solution for 1 h at room temperature. The IDA-Cu functionalized glyoxyl agarose support was recovered by filtration and washed with distilled water (10 volumes).

The protocol for the preparation of the IDA-Cu functionalized epoxy-based supports (Sepabeads® EC-EP/S R and Relizyme™ EP403/S R) was based on the method described by Tamayo-Cabezas & Karboune (2020, Process Biochemistry, 98: 11-20). The dry support (1:6 w/v) was suspended in an IDA solution (1.8M; pH of 11) and incubated for 5 h at room temperature at 55 rpm. The mixture was filtered and washed with distilled water (8 volumes). A solution of CuSO₄ (46 g/L; 30 ml) was then added and the mixture was incubated for 2 h at room temperature at 55 rpm. Finally, the support was filtered and initially washed with distilled water (8 volumes) then with MOPS (0.02M; pH of 6; 5 volumes).

The optimal pre-immobilization treatment and immobilization time were determined by suspending the glyoxyl agarose/TEA, glyoxyl agarose/IDA, or glyoxyl agarose/IDA-Cu (200 g/L) in potassium phosphate buffer (600 mM; pH of 6.8) containing G. oxydans LS1. Each support mixture (0.5 ml) was added to the G. oxydans LS1 solution (1 ml; 1 g/L) in order to immobilize 10 mg of protein per g of support. These mixtures were carried out in triplicates alongside blanks, where the buffer substituted the support into the LS solution, and were incubated at 4° C. and 80 rpm. The mixtures were centrifuged at 4 different time points (0, 24, 48, and 72 h) in order to take a 100 μl aliquot. The protein content of these aliquots was determined as previously described and based on the percent difference in protein content over time, the pre-immobilization treatment with IDA-Cu support after 5 h of immobilization time were selected for future immobilization.

G. oxydans LS1 and V. natriegens LS2 were respectively immobilized for 5 h into the 3 selected supports (Glyoxyl agarose/IDA-Cu, Sepabeads® EC-EP/IDA-Cu, and Relizyme™ EP403/IDA-Cu). The immobilized LSs were recovered by centrifugation for 2 min at 8,600 rpm and then the supernatants were removed while the supports were left to be washed and resuspended with potassium phosphate buffer (50 mM; pH of 6; 10 volumes). The protein content of the blanks, supernatants, and washes were quantified to determine the immobilization protein yield; while the retained activity was estimated from the specific activity of free and immobilized LS.

$$Immobilization Protein Yield = \frac{\begin{matrix}Free Enzyme Solution Protein Content - \\ ( Supernatant + Wash Protein Content )\end{matrix}}{Free Enzyme Solution Protein Content} \times 100$$

$$Retained Activity ( \% ) = \frac{Immobilized Enzyme Specific Activity}{Free Enzyme Specific Activity} \times 100$$

The free enzyme solutions as blanks were exposed to the same conditions as the mixtures with the supports and thus any loss of activity resulting from external forces was accounted for.

The reaction selectivity was assessed in the presence of sucrose alone or with lactose as substrates using G. oxydans LS1 and V. natriegens LS2 immobilized on Glyoxyl agarose/IDA-Cu, Sepabeads® EC-EP/IDA-Cu, and Relizyme™ EP403/IDA-Cu. After the enzymatic biotransformation reactions, the remaining sucrose and lactose as well as the released galactose, glucose, and fructose were quantified by HPAEC using a Dionex ICS-3000 system equipped with a PAD and a CarboPac PA20 column as described above. The hydrolysis was quantified from the concentration of released fructose while the transfructosylation was estimated based on the difference between the concentrations of fructose and glucose.

The Relizyme™ EP403/IDA-Cu immobilized G. oxydans LS1 and V. natriegens LS2 (1% v/v) were separately suspended in a high pH sodium phosphate buffer (100 mM; pH of 8.5) and incubated for 24 h at 18° C. and 80 rpm. This post-immobilization treatment was carried out in triplicates alongside the blanks, where the immobilized LSs were suspended in neutral pH potassium phosphate buffer (600 mM; pH of 6.8). The high pH-treated immobilized LSs were recovered upon centrifugation for 2 min at 8,600 rpm, washed and resuspended in potassium phosphate buffer (50 mM; pH of 6; 10 volumes).

PEI solution (0.1% v/v) was added to the high pH-treated immobilized LSs and incubated overnight at 4° C. and 80 rpm. The treatment was carried out in triplicates along with the blanks which were suspended in potassium phosphate buffer (50 mM; pH of 6) instead of the PEI treatment. The PEI-treated immobilized LSs were recovered upon centrifugation for 2 min at 8,600 rpm, washed and resuspended in potassium phosphate buffer (50 mM; pH of 6; 10 volumes).

The protein content and activity of the blanks, supernatants, and washes were quantified to assess the immobilization protein yield and retained activity of the immobilization LSs upon post-immobilization treatments. The blanks ran in parallel to each treatment and were thus experiencing the same conditions as the treated supports and thus any loss of activity resulting from external forces was taken in account.

To assess the thermal stability, free as well as high pH and PEI-treated immobilized G. oxydans LS1 and V. natriegens LS2 on Relizyme™ EP403/IDA-Cu in potassium phosphate buffer (50 mM; pH of 6) were incubated at 50° C. and 50 rpm. Aliquots were taken at selected incubation times, and the residual LS activities were quantified and compared to the initial ones.

To produce lactosucrose, the enzymatic biotransformation reactions were carried out using sucrose and lactose or lactose containing dairy by-products as substrates (WP and MP). Sucrose and lactose solutions were prepared at a ratio of 1:2 to reach a final concentration of 0.9M:0.45M. To initiate the biotransformation reactions, immobilized LS (5 U/ml) was added to the substrate solution. The biotransformation reactions were carried out at the optimal conditions for each immobilized LS on Relizyme™ EP403/IDA-Cu, corresponding to 30° C./pH 4 (50 mM ammonium acetate buffer) for immobilized G. oxydans LS1 and 45° C./pH 8 (50 mM ammonium bicarbonate buffer) for immobilized V. natriegens LS2. All reactions were done in duplicates under 50 rpm. A blank with no enzyme was run in parallel for each reaction. The biotransformation reactions were carried out over a time course of 24 h where aliquots were taken, placed in boiling water for 3 min to stop the reaction, and then stored at −20° C. until further analysis.

The end-products of the biotransformation reactions were analyzed using an Agilent 1290 II liquid chromatography system coupled to an Agilent 6560-ion mobility Q-TOF-MS. The analytes were separated with an InfinityLab Poroshell 120 HILIC-Z column (2.1×100 mm, 2.7 μm). Mobile phase A was LC-MS grade water with 0.3% NH₄OH and mobile phase B was ACN with 0.3% NH₄OH. Flow rate was set at 0.4 ml/min with a column temperature of 35° C. The constructed gradient started off with 85% B (0.0 to 0.5 min) that had a linear decrease to 30% B (0.5 to 9.0 min) where it was held (9.0 to 13.0 min) and then increased to 85% B (13.0 to 15.0 min), followed by a 3 min post-run. The mass spectrometer was equipped with a Dual AJS ESI ion source operating in negative ionization mode. MS conditions for ESI were as follows: drying gas temperature of 150° C. and flow rate of 11 L/min, sheath gas temperature of 350° C. and flow rate of 12 L/min, pressure on the nebulizer of 30 psig, capillary voltage of 4000V, fragmentor voltage of 200V, skimmer voltage of 30V, and nozzle voltage of 2000V. Full scan MS data was recorded at mass-to-charge ratios (m/z) from 80 to 1100 at a scan rate of 2 spectra/s and was collected at both centroid and profile mode. Reference ions (m/z at 112.985587 and 1033.988109 for ESI-) were used for automatic mass recalibration of each acquired spectrum. The quantification was performed using Quantitative Analysis 10.0 from Agilent MassHunter Workstation Software. The samples were prepared by diluting them in 50:50 ACN:Water (v/v) with the addition of myo-inositol (5 ppm) to serve as an internal standard.

The effects of the biotransformation reaction parameters were investigated using response surface methodology (RSM). Free and Relizyme™ EP403/IDA-Cu immobilized V. natriegens LS2 were selected, and the WP dairy by-product was used as a lactose substrate. The investigated reaction parameters included the incubation time (3-15 h) and substrate ratio of lactose to sucrose (0.5-2) while the other conditions, such as enzyme units (5 U/ml), buffer (50 mM ammonium bicarbonate; pH of 8), and temperature (45° C.), were kept constant. A five-level, two variable central composite rotatable design was created using Design Expert® Software. The full designs consisted of 4 factorial points, 4 axial points, and 3 center points and the levels of the parameters were determined based on the preliminary trials. The amount produced (g/L) and yield (%) of lactosucrose, kestose, nystose, and fructosyl nystose were the quantified responses.

The reusability of the immobilized V. natriegens LS2 on Relizyme™ EP403/IDA-Cu was evaluated by performing 6 consecutive biotransformation reactions using the same immobilized V. natriegens LS2 under the determined optimal conditions. After the appropriate reaction time, the immobilized V. natriegens LS2 was recovered and washed with ammonium bicarbonate buffer (50 mM; pH of 8) before it was reused under the same optimal conditions. The amount of produced lactosucrose (g/L) was quantified at each run.

### Example III

### Acceptor Specificity of Phenolic Compounds

5 U/mL of levansucrase (LS) was incubated with 0.9 M sucrose and 0.03 M acceptor molecules (phenolic compounds: Catechin, Epicatechin, Gallic acid and Caffeic acid—present in chocolate) at 10% DMSO at optimal temperature and pH of the selected LSs. LS1 from G. oxydans (strain 621H) and LS4 from B. graminis C4D1M were incubated at 30° C. in an ammonium acetate buffer of pH 4. LS2 from V. natriegens NBRC 15636 and LS3 from N. aromaticivorans were incubated at 45° C. in an ammonium bicarbonate buffer of pH 8 and 6 respectively. All reactions were done in duplicates under 50 rpm. The biotransformation reactions were carried out over a time course of 48 h where aliquots were taken, placed in boiling water for 5 min to stop the reaction, and then stored at −20° C. until further analysis.

The bioconversion of phenolic acceptors was analyzed via HPLC. The separation was performed on an Agilent Zobrax SB-C18 reversed-phase column (250 mm×4.6 mm, 5 μm), using a Beckman HPLC system equipped with an autosampler (Model 508), a UV/VIS DAD (Model 168) with computerized data handling and integration analysis (32 Karat, version 8). The samples were prepared by diluting them in 10:90 Acetonitrile:Water (v/v). They were analyzed using either of the two following gradient of water/formic acid 0.05% (v/v) and acetonitrile/formic acid 0.05% (v/v) (90/10 at 0 min, 50/50 at 20 min, 5/95 at 20.1 min and 90/10 at 35 min) or (90/10 at 0 min, 5/95 at 5 min, 90/10 at 25 min and 90/10 at 30 min) at a flow rate of 0.700 mL/min. The different phenolic compounds were quantified using UV detection at 254 nm. The bioconversion yield was based on the difference between the initial and final concentrations of phenolic compounds as a percentage of initial concentration of phenolic compounds.

The reaction mixtures were analyzed by LC-MS using an Agilent 1290 Infinity II LC system coupled to the 6560 ion mobility Q-TOF-MS (Agilent Technologies, Santa Clara, USA). The LC separation was conducted on a Poreshell120 EC-C18 analytical column (Agilent Technologies; 2.7 μm×3 mm×100 mm) connected with a Poreshell120 EC-C18 guard column (Agilent Technologies; 2.7 μm×3 mm×5 mm). The mobile phase A was HPLC water with 0.1% formic acid and the mobile phase B was acetonitrile with 0.1% formic acid. HPLC parameters were as follows: injection volume was 4 μL, the flow rate was 0.3 ml/min and the column temperature was set to 30° C. The mobile phase profile used for the run in negative ion mode was 2% B (0 to 1.0 min), 2%-20% B (1.0 to 4.0 min), 20%-100% B (4.0 to 8.0 min), 100% B (4.0 to 8.0 min), hold at 100% B (8.0-13.0 min), decrease to 2% B (13.0.0 to 13.5 min) and hold 2% B (13.5 to 14 min). The mass spectrometer was equipped with a Dual AJS ESI ion source operating in negative ionization mode. MS conditions were as follows: for ESI-, the drying gas temperature was 200° C., drying gas flow rate was 12 l/min, sheath gas temperature was 250° C., sheath gas flow rate was 12 L/min, the pressure on the nebulizer was 35 psi, the capillary voltage was 4000 V, the fragmentor voltage was 240 V, and the nozzle voltage was 1000 V. Full scan MS data were recorded between mass-to-charge ratios (m/z) 100 and 1700 at a scan rate of 2 spectra/s, and were collected at both centroid and profile mode. Reference ions (m/z at 112.9856 and 1033.9881 for ESI-) were used for automatic mass recalibration of each acquired spectrum. Data treatment was conducted using Quantitative Analysis B.07.01 from Agilent MassHunter Workstation Software.

The sucrose concentration and end-products profiles of fructooligosaccharides were characterized using an Agilent 1290 II liquid chromatography system coupled to an Agilent 6560-ion mobility Q-TOF-MS. The samples were prepared by diluting them in 50:50 Acetonitrile:Water (v/v) with the addition of myo-inositol (5 ppm) to serve as an internal standard. The analytes were separated with an InfinityLab Poroshell 120 HILIC-Z column (2.1×100 mm, 2.7 μm). Mobile phase A was LC-MS grade water with 0.3% NH₄OH and mobile phase B was acetonitrile with 0.3% NH₄OH. The flow rate was set at 0.4 ml/min with a column temperature of 35° C. The constructed gradient started off with 85% B (0.0 to 0.5 min) that had a linear decrease to 30% B (0.5 to 9.0 min) where it was held (9.0 to 13.0 min) and then increased to 85% B (13.0 to 15.0 min), followed by a 3 min post-run. The mass spectrometer was equipped with a Dual AJS ESI ion source operating in negative ionization mode. MS conditions for ESI was as follows: drying gas temperature of 150° C. and flow rate of 11 L/min, sheath gas temperature of 350° C. and flow rate of 12 L/min, pressure on the nebulizer of 30 psig, capillary voltage of 4000V, fragmentor voltage of 200V, skimmer voltage of 30V, and nozzle voltage of 2000V. Full scan MS data was recorded at mass-to-charge ratios (m/z) from 80 to 1100 at a scan rate of 2 spectra/s and will be collected at both centroid and profile mode. Reference ions (m/z at 112.985587 and 1033.988109 for ESI-) were used for automatic mass recalibration of each acquired spectrum. The quantification was performed using Quantitative Analysis 10.0 from Agilent MassHunter Workstation Software.

### Example IV

### Effect of Temperature on Lactosucrose Formation

5 U/mL of levansucrase (LS) was incubated with 0.9 M sucrose and 0.45 M lactose at pH 6.6 (pH of milk), at different temperatures (10° C., 30° C., 45° C.). All reactions were done in duplicates under 50 rpm. The biotransformation reactions were carried out over a time course of 24 h where aliquots were taken, placed in boiling water for 5 min to stop the reaction, and then stored at −20° C. until further analysis.

The sucrose concentration and end-products profiles of fructooligosaccharides were characterized using an Agilent 1290 II liquid chromatography system coupled to an Agilent 6560-ion mobility Q-TOF-MS. The samples were prepared by diluting them in 50:50 Acetonitrile:Water (v/v) with the addition of myo-inositol (5 ppm) to serve as an internal standard. The analytes were separated with an InfinityLab Poroshell 120 HILIC-Z column (2.1×100 mm, 2.7 μm). Mobile phase A was LC-MS grade water with 0.3% NH₄OH and mobile phase B was acetonitrile with 0.3% NH₄OH. The flow rate was set at 0.4 ml/min with a column temperature of 35° C. The constructed gradient started off with 85% B (0.0 to 0.5 min) that had a linear decrease to 30% B (0.5 to 9.0 min) where it was held (9.0 to 13.0 min) and then increased to 85% B (13.0 to 15.0 min), followed by a 3 min post-run. The mass spectrometer was equipped with a Dual AJS ESI ion source operating in negative ionization mode. MS conditions for ESI was as follows: drying gas temperature of 150° C. and flow rate of 11 L/min, sheath gas temperature of 350° C. and flow rate of 12 L/min, pressure on the nebulizer of 30 psig, capillary voltage of 4000V, fragmentor voltage of 200V, skimmer voltage of 30V, and nozzle voltage of 2000V. Full scan MS data was recorded at mass-to-charge ratios (m/z) from 80 to 1100 at a scan rate of 2 spectra/s and will be collected at both centroid and profile mode. Reference ions (m/z at 112.985587 and 1033.988109 for ESI-) were used for automatic mass recalibration of each acquired spectrum. The quantification was performed using Quantitative Analysis 10.0 from Agilent MassHunter Workstation Software.

### Example V

### Effect of Milk and Cocoa Powder

5 U/mL of levansucrase (LS) was incubated with different concentrations of sucrose (0.1 M and 0.3 M), milk powder (12.5% w/v and 20% w/v) and cocoa powder (1.428% w/v and 2.856% w/v) at optimal temperature and pH of the selected LSs. All reactions were done in duplicates under 50 rpm. The biotransformation reactions were carried out over a time course of 24 h where aliquots were taken, placed in boiling water for 5 min to stop the reaction, and then stored at −20° C. until further analysis.

The end-products profiles of fructooligosaccharides were characterized using an Agilent 1290 II liquid chromatography system coupled to an Agilent 6560-ion mobility Q-TOF-MS. The samples were prepared by diluting them in 50:50 Acetonitrile:Water (v/v) with the addition of myo-inositol (5 ppm) to serve as an internal standard. The analytes were separated with an InfinityLab Poroshell 120 HILIC-Z column (2.1×100 mm, 2.7 μm). Mobile phase A was LC-MS grade water with 0.3% NH₄OH and mobile phase B was acetonitrile with 0.3% NH₄OH. The flow rate was set at 0.4 ml/min with a column temperature of 35° C. The constructed gradient started off with 85% B (0.0 to 0.5 min) that had a linear decrease to 30% B (0.5 to 9.0 min) where it was held (9.0 to 13.0 min) and then increased to 85% B (13.0 to 15.0 min), followed by a 3 min post-run. The mass spectrometer was equipped with a Dual AJS ESI ion source operating in negative ionization mode. MS conditions for ESI was as follows: drying gas temperature of 150° C. and flow rate of 11 L/min, sheath gas temperature of 350° C. and flow rate of 12 L/min, pressure on the nebulizer of 30 psig, capillary voltage of 4000V, fragmentor voltage of 200V, skimmer voltage of 30V, and nozzle voltage of 2000V. Full scan MS data was recorded at mass-to-charge ratios (m/z) from 80 to 1100 at a scan rate of 2 spectra/s and will be collected at both centroid and profile mode. Reference ions (m/z at 112.985587 and 1033.988109 for ESI-) were used for automatic mass recalibration of each acquired spectrum. The quantification was performed using Quantitative Analysis 10.0 from Agilent MassHunter Workstation Software.

While the description has been described in connection with specific embodiments thereof, it will be understood that it is capable of further modifications and this application is intended to cover any variations, uses, or adaptations and including such departures from the present disclosure as come within known or customary practice within the art and as may be applied to the essential features hereinbefore set forth, and as follows in the scope of the appended claims.

What is claimed is:

1. A process for producing lactosucrose from a dairy starting material, the process comprising contacting a source of lactose and a source of sucrose with a levansucrase (LS) selected from Bacillus amyloliquefaciens (ATCC 23350), Gluconobacter oxydans (strain 621H) (LS1), Vibrio natriegens NBRC 15636 (LS2), Novosphingobium aromaticivorans (LS3), and Burkholderia graminis C4D1M (LS4), or a combination thereof, producing lactosucrose.

2. The process of claim 1, wherein the LS is Bacillus amyloliquefaciens (ATCC 23350), Gluconobacter oxydans (strain 621H) (LS1), Vibrio natriegens NBRC 15636 (LS2), or a combination thereof.

3. The process of claim 1, wherein the LS is Vibrio natriegens NBRC 15636 (LS2).

4. The process of claim 1, further comprising producing fructooligosaccharides (FOSs).

5. The process of claim 4, wherein the FOSs are kestose, nystose, fructosyl nystose, or a combination thereof.

6. The process of 1, wherein the levansucrase further transfructosylate phenolic compounds from the dairy starting material.

7. The process of claim 6, wherein the phenolic compounds are catechin, gallic acid, caffeic acid, or epicatechin.

8. The process of claim 1, further producing oligomers and polysaccharides.

9. The process of claim 1, wherein the source of lactose is a dairy product.

10. The process of claim 9, wherein the dairy product is a lactose enriched mixture, a whey permeate (WP), or a milk permeate (MP).

11. The process of claim 10, wherein the dairy starting material is milk, smoothie, yogurt, high protein dairy mixture, ice cream, flavored milk or chocolate milk.

12. The process of claim 1, wherein the source of sucrose is white sugar, maple syrup, or a combination thereof.

13. The process of claim 1, wherein the LS is immobilized on a solid support.

14. The process of claim 13, wherein the solid support is a glyoxyl agarose support.

15. The process of claim 14, wherein the glyoxyl agarose support is further modified with iminodiacetic acid (IDA), IDA-Cu, and triethylamine (TEA).

16. The process of claim 15, wherein the solid support is Relizyme™ EP403 or Sepabeads® EC-EP/S.

17. The process of claim 13, wherein the LS is stabilized post immobilization on the solid support by exposing the LS immobilized on the support to high the pH conditions.

18. The process of claim 13, wherein the LS immobilized on the support further comprises a polyaminated crosslinker (PEI).

19. A kit for producing lactosucrose from a dairy starting material as defined in claim 1 comprising a solid support and a levansucrase (LS) selected from Gluconobacter oxydans (strain 621H) (LS1), Vibrio natriegens NBRC 15636 (LS2), Novosphingobium aromaticivorans (LS3), and Burkholderia graminis C4D1M (LS4), or a combination thereof.

20. An enriched dairy product comprising a levansucrase (LS) selected from Gluconobacter oxydans (strain 621H) (LS1), Vibrio natriegens NBRC 15636 (LS2), Novosphingobium aromaticivorans (LS3), and Burkholderia graminis C4D1M (LS4), or a combination thereof and a source of sucrose.
